(19) United States
(12) Patent Application Publication
(10) Pub. No.: US 2026/0226563 A1
(43) Pub. Date: Aug. 6, 2026

(54) COMPOSITIONS AND METHODS FOR DETECTING STX NUCLEIC ACIDS

(71) Applicant: Gen-Probe Incorporated, San Diego, CA (US)

(72) Inventors: Pasi M.J. PIIPARINEN, Siuntio (FI); Minna M. MAUNULA, Helsinki (FI)

(21) Appl. No.: 19/151,258

(22) PCT Filed: Jan. 29, 2024
(86) PCT No.: PCT/IB2024/050810

§ 371 Date: Jul. 25, 2025

(30) Foreign Application Priority Data
Jan. 31, 2023 (WO) PCT/IB2023/050846

(51) Int. Cl. C12Q 1/689

(52) U.S. Cl. CPC C12Q 1/689; C12Q 2600/156; C12Q 2600/16

(57) ABSTRACT

Disclosed are nucleic acid oligomers, including amplification oligomers, detection probes, and combinations thereof, for detection of one or more gastrointestinal pathogens expressing shiga toxin (stx). Also disclosed are methods of specific nucleic acid amplification and detection, including multiplex assays, using the disclosed oligomers, as well as corresponding reaction mixtures and kits.

### CROSS-REFERENCE(S) TO RELATED APPLICATION(S)

This application claims priority to International Application No. PCT/IB2023/050846, filed Jan. 31, 2023, which is incorporated by reference herein in its entirety.

### SEQUENCE LISTING

The present application is filed with a Sequence Listing in electronic format. The Sequence Listing is provided as a file entitled “MOBID9PCT2_Sequence_Listing.xml” created on Jan. 29, 2024, which is 84 kilobytes in size. The information in the electronic format of the sequence listing is incorporated herein by reference in its entirety.

### BACKGROUND

Bacterial gastroenteritis is inflammation of the stomach and intestines that results in acute diarrhea (3 or more episodes per day) lasting less than 14 days and may also include symptoms such as nausea, vomiting, and abdominal cramping. See Thielman and Guerrant, The New England Journal of Medicine, 350:38-47, 2004. In the United States it is estimated that there are >200 million cases of diarrheal illness per year resulting in 73 million physician consultations, 1.8 million hospitalizations, and up to 6000 deaths. See Thielman and Guerrant, supra; Guerrant et al., Clinical Infectious Diseases, 32:331-350, 2001. Certain bacteria, including Escherichia and Shigella, produce a toxin called Shiga toxin (stx) that can cause gastroenteritis. In some cases, the gastroenteritis can develop into hemolytic uremic syndrome (HUS). See Smith et al., Adv. Appl. Microbiol., 28:145-197, 2014. The populations most at risk due to bacterial gastroenteritis infection are children (<5), the elderly, and immunocompromised. Infection, however, can occur in all age groups. The mode of infection is via the fecal-oral route typically from ingesting contaminated food or water or as a result of poor hygiene (hand-washing).

Shiga Toxin-Producing Escherichia coli (STEC), for example, can produce Stx1 or Stx2 virulence factors that can cause diarrhea and, possibly, hemorrhagic colitis. See Bai et al., Microorganisms, 9(2374), 2021. Stx1 and Stx2 target human podocytes (visceral epithelial cells) and renal microvascular endothelial cells, where they inhibit protein synthesis. See Obrig, Toxins, 2:2769-2794, 2010. E. coli Stx2 can be divided into subtypes or variants, e.g., Stx2a, Stx2b, Stx2c, Stx2d, Stx2e, Stx2f, Stx2g, Stx2h, Stx2i, Stx2j, Stx2k, Stx21, and Stx2m; which differ in receptor binding, toxin potency and host specificity. See Bai et al., Microorganisms, 9(2374), 2021. The Stx2e and Stx2f variants, in particular, have been related to severe clinical symptoms such as HUS. Id. Some of the variants, e.g., Stxh, Stxi, Stxj, Stxk, Stx1, and Stxm, have been identified only recently (Id.) and may escape detection by existing diagnostic methods. Due to the growing number of Stx variants, there is a need to efficiently and sensitively detect their presence in samples, including biological specimens to provide diagnostic and prognostic information to physicians treating patients suffering from, or suspected of suffering from, bacterial gastroenteritis or related disorders.

### SUMMARY

A sample containing or suspected of containing bacteria capable of producing shiga toxin (stx) variants may be analyzed using the methods, materials, assays, and kits disclosed herein. The following embodiments are among those provided by the disclosure.

Embodiment 1 is a multiplex method for determining the presence or absence one or more variants of a stx target nucleic acid in a sample, said method comprising:
a. contacting a sample, said sample suspected of containing a stx target nucleic acid with a plurality of stx-specific amplification oligomers for amplifying a target region of the stx target nucleic acid, wherein:
   i. the plurality of stx-specific amplification oligomers comprises first, second, third, fourth, fifth, and sixth oligomers respectively comprising target-hybridizing sequences substantially corresponding to nucleotide sequences of: (i) SEQ ID NO: 29; (ii) SEQ ID NO: 5; (iii) SEQ ID NO: 23; (iv) SEQ ID NO: 26; (v) SEQ ID NO: 33; and (vi) SEQ ID NO: 40;
   ii. the plurality of stx-specific amplification oligomers comprises first, second, third, fourth, and fifth oligomers respectively comprising target-hybridizing sequences substantially corresponding to nucleotide sequences of: (i) SEQ ID NO: 14; (ii) SEQ ID NO: 15; (iii) SEQ ID NO: 18; (iv) SEQ ID NO: 30; and (v) SEQ ID NO: 20;
   iii. the plurality of stx-specific amplification oligomers comprises first, second, third, fourth, and fifth oligomers respectively comprising target-hybridizing sequences substantially corresponding to nucleotide sequences of: (i) SEQ ID NO: 21; (ii) SEQ ID NO: 19; (iii) SEQ ID NO: 18; (iv) SEQ ID NO: 30; and (v) SEQ ID NO: 20;
   iv. the plurality of stx-specific amplification oligomers comprises first, second, third, fourth, and fifth oligomers respectively comprising target-hybridizing sequences substantially corresponding to nucleotide sequences of: (i) SEQ ID NO: 31 and; (ii) SEQ ID NO: 27; (iii) SEQ ID NO: 18; (iv) SEQ ID NO: 30; and (v) SEQ ID NO: 20;
   v. the plurality of stx-specific amplification oligomers comprises first, second, third, fourth, and fifth oligomers respectively comprising target-hybridizing sequences substantially corresponding to nucleotide sequences of: (i) SEQ ID NO: 36; (ii) SEQ ID NO: 27; (iii) SEQ ID NO: 18; (iv) SEQ ID NO: 30; and (v) SEQ ID NO: 20; or
   vi. the plurality of stx-specific amplification oligomers comprises first, second, third, fourth, fifth, sixth, and seventh oligomers respectively comprising target-hybridizing sequences substantially corresponding to nucleotide sequences of: (i) SEQ ID NO: 3; (ii) SEQ ID NO: 4; (iii) SEQ ID NO: 18; (iv) SEQ ID NO: 30; (v) SEQ ID NO: 20; (vi) SEQ ID NO: 1; and (vii) SEQ ID NO: 2;
b. performing an in vitro nucleic acid amplification reaction, wherein the stx target nucleic acid, if present in said sample, is used as a template for generating one or more amplification products corresponding to the stx target regions; and
c. (i) determining the sequences of the one or more amplification products, or (ii) detecting the presence or absence of the one or more amplification products using a plurality of detection probes, wherein the detection probes are specific for one or more variants of the stx target nucleic acid,
thereby determining the presence or absence of the one or more variants of the stx target nucleic acid in said sample.

Embodiment 2 is the method of embodiment 1, wherein the plurality of stx-specific amplification oligomers further comprises first, second, third, and fourth additional stx-specific amplification oligomers for amplifying the target region of the stx target nucleic acid, wherein the first, second, third, and fourth additional stx-specific amplification oligomers respectively comprise first, second, third, and fourth target-hybridizing sequences substantially corresponding to nucleotide sequences of: (i) SEQ ID NO: 37; (ii) SEQ ID NO: 6; (iii) SEQ ID NO: 24; and (iv) SEQ ID NO: 41.

Embodiment 3 is the method of embodiment 1 or 2, wherein the plurality of detection probes comprises (i) a first detection probe comprising a target-hybridizing sequence substantially corresponding to the nucleotide sequence of SEQ ID NO: 28 or 17; or (ii) a first detection probe comprising a target-hybridizing sequence substantially corresponding to the nucleotide sequence of SEQ ID NO: 28 and an additional detection probe comprising a target-hybridizing sequence substantially corresponding to the nucleotide sequence of SEQ ID NO: 17.

Embodiment 4 is the method of embodiment 3, wherein the first detection probe comprises the nucleotide sequence of SEQ ID NO: 10.

Embodiment 5 is the method of any one of embodiments 3-4, wherein the plurality of detection probes comprises a second detection probe comprising a target-hybridizing sequence substantially corresponding to the nucleotide sequence of SEQ ID NO: 22; a third detection probe comprising a target-hybridizing sequence substantially corresponding to the nucleotide sequence of SEQ ID NO: 39; a fourth detection probe comprising a target-hybridizing sequence substantially corresponding to the nucleotide sequence of SEQ ID NO: 16; a fifth detection probe comprising a target-hybridizing sequence substantially corresponding to the nucleotide sequence of SEQ ID NO: 42; a sixth detection probe comprising a target-hybridizing sequence substantially corresponding to the nucleotide sequence of SEQ ID NO: 38; and/or a seventh detection probe comprising a target-hybridizing sequence substantially corresponding to the nucleotide sequence of SEQ ID NO: 25.

Embodiment 6 is the method of embodiment 5, wherein the second detection probe comprises the nucleotide sequence of SEQ ID NO: 8; the third detection probe comprises the nucleotide sequence of SEQ ID NO: 12; the fourth detection probe comprises the nucleotide sequence of SEQ ID NO: 7; the fifth detection probe comprises the nucleotide sequence of SEQ ID NO: 13; the sixth detection probe comprises the nucleotide sequence of SEQ ID NO: 11; and/or the seventh detection probe comprises the nucleotide sequence of SEQ ID NO: 9.

Embodiment 7 is the method of any one of embodiments 1-6, wherein the plurality of stx-specific amplification oligomers comprises first, second, third, fourth, fifth, and sixth oligomers respectively comprising target-hybridizing sequences substantially corresponding to nucleotide sequences of: (i) SEQ ID NO: 29; (ii) SEQ ID NO: 5; (iii) SEQ ID NO: 23; (iv) SEQ ID NO: 26; (v) SEQ ID NO: 33; and (vi) SEQ ID NO: 40.

Embodiment 8 is the method of any one of embodiments 1-6, wherein the plurality of stx-specific amplification oligomers comprises first, second, third, fourth, and fifth oligomers respectively comprising target-hybridizing sequences substantially corresponding to nucleotide sequences of: (i) SEQ ID NO: 14; (ii) SEQ ID NO: 15; (iii) SEQ ID NO: 18; (iv) SEQ ID NO: 30; and (v) SEQ ID NO: 20.

Embodiment 9 is the method of any one of embodiments 1-6, wherein the plurality of stx-specific amplification oligomers comprises first, second, third, fourth, and fifth oligomers respectively comprising target-hybridizing sequences substantially corresponding to nucleotide sequences of: (i) SEQ ID NO: 21; (ii) SEQ ID NO: 19; (iii) SEQ ID NO: 18; (iv) SEQ ID NO: 30; and (v) SEQ ID NO: 20.

Embodiment 10 is the method of any one of embodiments 1-6, wherein the plurality of stx-specific amplification oligomers comprises first, second, third, fourth, and fifth oligomers respectively comprising target-hybridizing sequences substantially corresponding to nucleotide sequences of: (i) SEQ ID NO: 31 and; (ii) SEQ ID NO: 27; (iii) SEQ ID NO: 18; (iv) SEQ ID NO: 30; and (v) SEQ ID NO: 20.

Embodiment 11 is the method of any one of embodiments 1-6, wherein the plurality of stx-specific amplification oligomers comprises first, second, third, fourth, and fifth oligomers respectively comprising target-hybridizing sequences substantially corresponding to nucleotide sequences of: (i) SEQ ID NO: 36; (ii) SEQ ID NO: 27; (iii) SEQ ID NO: 18; (iv) SEQ ID NO: 30; and (v) SEQ ID NO: 20.

Embodiment 12 is the method of any one of embodiments 1-6, wherein the plurality of stx-specific amplification oligomers comprises first, second, third, fourth, fifth, sixth, and seventh oligomers respectively comprising target-hybridizing sequences substantially corresponding to nucleotide sequences of: (i) SEQ ID NO: 3; (ii) SEQ ID NO: 4; (iii) SEQ ID NO: 13; (iv) SEQ ID NO: 30; (v) SEQ ID NO: 20; (vi) SEQ ID NO: 1; and (vii) SEQ ID NO: 2.

Embodiment 13 is the method of embodiment 7, wherein the nucleotide sequences of the first, second, third, fourth, fifth, and sixth oligomers respectively comprise or consist of: (i) SEQ ID NO: 29; (ii) SEQ ID NO: 5; (iii) SEQ ID NO: 23; (iv) SEQ ID NO: 26; (v) SEQ ID NO: 33; and (vi) SEQ ID NO: 40.

Embodiment 14 is the method of embodiment 8, wherein the nucleotide sequences of the first, second, third, fourth, and fifth oligomers respectively comprise or consist of: (i) SEQ ID NO: 14; (ii) SEQ ID NO: 15; (iii) SEQ ID NO: 18; (iv) SEQ ID NO: 30; and (v) SEQ ID NO: 20.

Embodiment 15 is the method of embodiment 9, wherein the nucleotide sequences of the first, second, third, fourth, and fifth oligomers respectively comprise or consist of: (i) SEQ ID NO: 21; (ii) SEQ ID NO: 19; (iii) SEQ ID NO: 18; (iv) SEQ ID NO: 30; and (v) SEQ ID NO: 20.

Embodiment 16 is the method of embodiment 10, wherein the nucleotide sequences of the first, second, third, fourth, and fifth oligomers respectively comprise or consist of: (i) SEQ ID NO: 31 and; (ii) SEQ ID NO: 27; (iii) SEQ ID NO: 18; (iv) SEQ ID NO: 30; and (v) SEQ ID NO: 20.

Embodiment 17 is the method of embodiment 11, wherein the nucleotide sequences of the first, second, third, fourth, and fifth oligomers respectively comprise or consist of: (i) SEQ ID NO: 36; (ii) SEQ ID NO: 27; (iii) SEQ ID NO: 18; (iv) SEQ ID NO: 30; and (v) SEQ ID NO: 20.

Embodiment 18 is the method of embodiment 12, wherein the nucleotide sequences of the first, second, third, fourth, fifth, sixth, and seventh oligomers respectively comprise or consist of: (i) SEQ ID NO: 3; (ii) SEQ ID NO: 4; (iii) SEQ ID NO: 18; (iv) SEQ ID NO: 30; (v) SEQ ID NO: 20; (vi) SEQ ID NO: 1; and (vii) SEQ ID NO: 2.

Embodiment 19 is the method of any one of embodiments 1-18, wherein the plurality of detection probes comprises a first detection probe comprising a target-hybridizing sequence substantially corresponding to a nucleotide sequence of SEQ ID NO: 28; a second detection probe comprising a target-hybridizing sequence substantially corresponding to a nucleotide sequence of SEQ ID NO: 22; a third detection probe comprising a target-hybridizing sequence substantially corresponding to a nucleotide sequence of SEQ ID NO: 39; a fourth detection probe comprising a target-hybridizing sequence substantially corresponding to a nucleotide sequence of SEQ ID NO: 16; a fifth detection probe comprising a target-hybridizing sequence substantially corresponding to a nucleotide sequence of SEQ ID NO: 42; a sixth detection probe comprising a target-hybridizing sequence substantially corresponding to a nucleotide sequence of SEQ ID NO: 38; and a seventh detection probe comprising a target-hybridizing sequence substantially corresponding to a nucleotide sequence of SEQ ID NO: 25.

Embodiment 20 is the method of any one of embodiments 1-18, wherein the plurality of detection probes comprises a first detection probe comprising a target-hybridizing sequence substantially corresponding to a nucleotide sequence of SEQ ID NO: 17; a second detection probe comprising a target-hybridizing sequence substantially corresponding to a nucleotide sequence of SEQ ID NO: 22; a third detection probe comprising a target-hybridizing sequence substantially corresponding to a nucleotide sequence of SEQ ID NO: 39; a fourth detection probe comprising a target-hybridizing sequence substantially corresponding to a nucleotide sequence of SEQ ID NO: 16; a fifth detection probe comprising a target-hybridizing sequence substantially corresponding to a nucleotide sequence of SEQ ID NO: 42; a sixth detection probe comprising a target-hybridizing sequence substantially corresponding to a nucleotide sequence of SEQ ID NO: 38; and a seventh detection probe comprising a target-hybridizing sequence substantially corresponding to a nucleotide sequence of SEQ ID NO: 25.

Embodiment 21 is the method of embodiment 19, wherein the nucleotide sequence of the first detection probe comprises or consists of SEQ ID NO: 28; the nucleotide sequence of the second detection probe comprises or consists of SEQ ID NO: 22; the nucleotide sequence of the third detection probe comprises or consists of SEQ ID NO: 39; the nucleotide sequence of the fourth detection probe comprises or consists of SEQ ID NO: 16; the nucleotide sequence of the fifth detection probe comprises or consists of SEQ ID NO: 42; the nucleotide sequence of the sixth detection probe comprises or consists of SEQ ID NO: 38; and the nucleotide sequence of the seventh detection probe comprises or consists of SEQ ID NO: 25.

Embodiment 22 is the method of embodiment 20, wherein the nucleotide sequence of the first detection probe comprises or consists of SEQ ID NO: 17; the nucleotide sequence of the second detection probe comprises or consists of SEQ ID NO: 22; the nucleotide sequence of the third detection probe comprises or consists of SEQ ID NO: 39; the nucleotide sequence of the fourth detection probe comprises or consists of SEQ ID NO: 16; the nucleotide sequence of the fifth detection probe comprises or consists of SEQ ID NO: 42; the nucleotide sequence of the sixth detection probe comprises or consists of SEQ ID NO: 38; and the nucleotide sequence of the seventh detection probe comprises or consists of SEQ ID NO: 25.

Embodiment 23 is a method for testing a sample for the presence of pathogenic Escherichia coli, comprising the steps of:
a. contacting the sample with a plurality of stx-specific amplification oligomers for amplifying a target region of a stx target nucleic acid, wherein the plurality of stx-specific amplification oligomers comprises (i) at least one oligomer comprising a nucleic acid sequence selected from the group consisting of: SEQ ID NOs: 5, 6, 23, 26, 29, and 37, and (ii) an oligomer comprising a nucleic acid sequence consisting of SEQ ID NO: 43, wherein when N at position 1 is an adenine, then R is a guanine;
b. performing an in vitro nucleic acid amplification reaction, wherein the stx target nucleic acid, if present in said sample, is used as a template for generating one or more amplification products corresponding to the stx target region; and
c. detecting the presence or absence of the one or more amplification products thereby determining the presence or absence of the pathogenic Escherichia coli in the sample.

Embodiment 24 is the method of embodiment 23, wherein the oligomer at (ii) is selected from the group consisting of SEQ ID NOs:33 and 40.

Embodiment 25 is the method of embodiment 23 or 24, wherein the oligomer at (i) is selected from the group consisting of SEQ ID NOs:5, 23, 26, and 29.

Embodiment 26 is a method for testing a sample for the presence of pathogenic Escherichia coli, comprising the steps of:
a. contacting the sample with a plurality of stx-specific amplification oligomers for amplifying a target region of a stx target nucleic acid, wherein the plurality of stx-specific amplification oligomers, comprises:
   i. one or more oligomers comprising nucleic acid sequences selected from the group consisting of: SEQ ID NOs: 18, 20, and 30; and
   ii. one or more oligomers comprising nucleic acid sequences selected from the group consisting of: SEQ ID NOs: 1, 2, 3, 4, 14, 15, 19, 21, 26, 27, and 31;
b. performing an in vitro nucleic acid amplification reaction, wherein the stx target nucleic acid, if present in said sample, is used as a template for generating one or more amplification products corresponding to the stx target region; and
c. detecting the presence or absence of the one or more amplification products thereby determining the presence or absence of the pathogenic Escherichia coli in the sample.

Embodiment 27 is the method of embodiment 26, wherein the plurality of stx-specific amplification oligomers comprises (A) two oligomers respectively comprising nucleic acid sequences consisting of SEQ ID NOs: 14 and 15; (B) two oligomers respectively comprising nucleic acid sequences consisting of SEQ ID NOs: 19 and 21; (C) two oligomers respectively comprising nucleic acid sequences consisting of SEQ ID NOs: 27 and 31; (D) two oligomers respectively comprising nucleic acid sequences consisting of SEQ ID NOs: 26 and 31; or (E) four oligomers respectively comprising nucleic acid sequences consisting of SEQ ID NOs: 1, 2, 3, and 4.

Embodiment 28 is a method for testing a sample for the presence of pathogenic Escherichia coli, comprising the steps of:
a. contacting the sample with a plurality of stx-specific amplification oligomers for amplifying a target region of a stx target nucleic acid, wherein the plurality of stx-specific amplification oligomers, comprises:
   i. one or more oligomers comprising nucleic acid sequences selected from the group consisting of: SEQ ID NOs: 18, 20, and 30; and
   ii. one or more oligomers comprising nucleic acid sequences each individually selected from the group consisting of: (A) nucleic acid sequences that are from 18 to 22 contiguous nucleotides of SEQ ID NO: 44, and (B) nucleic acid sequences that are from 18 to 20 contiguous nucleotides of SEQ ID NO: 45;
b. performing an in vitro nucleic acid amplification reaction, wherein the stx target nucleic acid, if present in said sample, is used as a template for generating one or more amplification products corresponding to the stx target region; and
c. detecting the presence or absence of the one or more amplification products thereby determining the presence or absence of the pathogenic Escherichia coli in the sample.

Embodiment 29 is the method of embodiment 28, wherein the plurality of stx-specific amplification oligomers at a.ii. comprises (A) two oligomers respectively comprising nucleic acid sequences consisting of: SEQ ID NOs: 27 and 31; (B) two oligomers respectively comprising nucleic acid sequences consisting of SEQ ID NOs: 26 and 31; (C) two oligomers respectively comprising nucleic acid sequences consisting of SEQ ID NOs: 1 and 2; (D) two oligomers respectively comprising nucleic acid sequences consisting of SEQ ID NOs: 3 and 4; or (E) four oligomers respectively comprising nucleic acid sequences consisting of SEQ ID NOs: 1, 2, 3, and 4.

Embodiment 30 is the method of any one of embodiments 1-29, where the amplification reaction is a polymerase chain reaction (PCR).

Embodiment 31 is the method of embodiment 30, wherein the amplification reaction is a real-time polymerase chain reaction.

Embodiment 32 is the method of any one of embodiments 28-31, wherein detecting the presence or absence of the one or more amplification products comprises detecting the presence or absence of the one or more amplification products using a plurality of detection probes, wherein the detection probes are specific for one or more variants of the stx target nucleic acid.

Embodiment 33 is the method of any one of embodiments 1-27 or 32, wherein each of the plurality of detection probes comprises a fluorescent label.

Embodiment 34 is the method of embodiment 33, wherein each of the plurality of detection probes further comprises a quenching label.

Embodiment 35 is the method of any one of embodiments 1-27 or 32-34, wherein each of the plurality of detection probes is immobilized on a solid support.

Embodiment 36 is the method of embodiment 35, wherein the solid support is a microarray and detection comprises hybridization of amplified products to one or more probes immobilized on the microarray.

Embodiment 37 is the method of any one of embodiments 1-32 or 35-36, wherein one or more of the stx-specific amplification oligomers comprises a fluorescent label.

Embodiment 38 is the method of embodiment 37, wherein
a. two or more of the stx-specific amplification oligomers comprise a fluorescent label; or
b. each of the stx-specific amplification oligomers comprises a fluorescent label, wherein the stx-specific amplification oligomers comprise a sequence substantially corresponding to (i) SEQ ID NOs: 29, 5, 23, and 26; (ii) SEQ ID NOs: 14 and 15; (iii) SEQ ID NOs: 21 and 19; (iv) SEQ ID NOs: 31 and 27; (v) SEQ ID NO: 36; or (vi) SEQ ID NOs: 3, 4, 1, and 2; or
c. (i) SEQ ID NOs: 33 and 40; (ii) SEQ ID NOs: 30 and 20; or (iii) SEQ ID NOs: 18, 30, and 20 comprise a fluorescent label.

Embodiment 39 is the method of any one of embodiments 1-38, wherein step c comprises determining the sequences of the one or more amplification products.

Embodiment 40 is the method of any one of embodiments 1-39, wherein the sample comprises stool, food, or water.

Embodiment 41 is the method of any one of embodiments 1-40, further comprising the step of isolating nucleic acid from the sample, optionally wherein the isolating step is before step a.

Embodiment 42 is a set of oligonucleotides for determining the presence or absence one or more variants of a stx target nucleic acid in a sample, said oligonucleotide set comprising a plurality of stx-specific amplification oligomers for amplifying a target region of the stx target nucleic acid, wherein:
a. the plurality of stx-specific amplification oligomers comprises first, second, third, fourth, fifth, and sixth oligomers respectively comprising target-hybridizing sequences substantially corresponding to nucleotide sequences of (i) SEQ ID NO: 29; (ii) SEQ ID NO: 5; (iii) SEQ ID NO: 23; (iv) SEQ ID NO: 26; (v) SEQ ID NO: 33; and (vi) SEQ ID NO: 40;
b. the plurality of stx-specific amplification oligomers comprises first, second, third, fourth, and fifth oligomers respectively comprising target-hybridizing sequences substantially corresponding to nucleotide sequences of: (i) SEQ ID NO: 14; (ii) SEQ ID NO: 15; (iii) SEQ ID NO: 18; (iv) SEQ ID NO: 30; and (v) SEQ ID NO: 20;
c. the plurality of stx-specific amplification oligomers comprises first, second, third, fourth, and fifth oligomers respectively comprising target-hybridizing sequences substantially corresponding to nucleotide sequences of: (i) SEQ ID NO: 21; (ii) SEQ ID NO: 19; (iii) SEQ ID NO: 18; (iv) SEQ ID NO: 30; and (v) SEQ ID NO: 20;
d. the plurality of stx-specific amplification oligomers comprises first, second, third, fourth, and fifth oligomers respectively comprising target-hybridizing sequences substantially corresponding to nucleotide sequences of: (i) SEQ ID NO: 31 and; (ii) SEQ ID NO: 27; (iii) SEQ ID NO: 18; (iv) SEQ ID NO: 30; and (v) SEQ ID NO: 20;
e. the plurality of stx-specific amplification oligomers comprises first, second, third, fourth, and fifth oligomers respectively comprising target-hybridizing sequences substantially corresponding to nucleotide sequences of: (i) SEQ ID NO: 36; (ii) SEQ ID NO: 27; (iii) SEQ ID NO: 18; (iv) SEQ ID NO: 30; and (v) SEQ ID NO: 20; or
f. the plurality of stx-specific amplification oligomers comprises first, second, third, fourth, fifth, sixth, and seventh oligomers respectively comprising target-hybridizing sequences substantially corresponding to nucleotide sequences of: (i) SEQ ID NO: 3; (ii) SEQ ID NO: 4; (iii) SEQ ID NO: 18; (iv) SEQ ID NO: 30; (v) SEQ ID NO: 20; (vi) SEQ ID NO: 1; and (vii) SEQ ID NO: 2.

Embodiment 43 is the oligonucleotide set of embodiment 42, wherein the plurality of stx-specific amplification oligomers further comprises first, second, third, and fourth additional stx-specific amplification oligomers for amplifying the target region of the stx target nucleic acid, wherein the first, second, third, and fourth additional stx-specific amplification oligomers respectively comprise first, second, third, and fourth target-hybridizing sequences substantially corresponding to nucleotide sequences of: (i) SEQ ID NO: 37; (ii) SEQ ID NO: 6; (iii) SEQ ID NO: 24; and (iv) SEQ ID NO: 41.

Embodiment 44 is the oligonucleotide set of embodiment 42 or 43, wherein the plurality of detection probes comprises (i) a first detection probe comprising a target-hybridizing sequence substantially corresponding to the nucleotide sequence of SEQ ID NO: 28 or 17; or (ii) a first detection probe comprising a target-hybridizing sequence substantially corresponding to the nucleotide sequence of SEQ ID NO: 28 and an additional detection probe comprising a target-hybridizing sequence substantially corresponding to the nucleotide sequence of SEQ ID NO: 17.

Embodiment 45 is the oligonucleotide set of embodiment 44, wherein the first detection probe comprises the nucleotide sequence of SEQ ID NO: 10.

Embodiment 46 is the oligonucleotide set of any one of embodiments 42-45, wherein the plurality of detection probes comprises a second detection probe comprising a target-hybridizing sequence substantially corresponding to the nucleotide sequence of SEQ ID NO: 22; a third detection probe comprising a target-hybridizing sequence substantially corresponding to the nucleotide sequence of SEQ ID NO: 39; a fourth detection probe comprising a target-hybridizing sequence substantially corresponding to the nucleotide sequence of SEQ ID NO: 16; a fifth detection probe comprising a target-hybridizing sequence substantially corresponding to the nucleotide sequence of SEQ ID NO: 42; a sixth detection probe comprising a target-hybridizing sequence substantially corresponding to the nucleotide sequence of SEQ ID NO: 38; and/or a seventh detection probe comprising a target-hybridizing sequence substantially corresponding to the nucleotide sequence of SEQ ID NO: 25.

Embodiment 47 is the oligonucleotide set of embodiment 46, wherein the second detection probe comprises the nucleotide sequence of SEQ ID NO: 8; the third detection probe comprises the nucleotide sequence of SEQ ID NO: 12; the fourth detection probe comprises the nucleotide sequence of SEQ ID NO: 7; the fifth detection probe comprises the nucleotide sequence of SEQ ID NO: 13; the sixth detection probe comprises the nucleotide sequence of SEQ ID NO: 11; and/or the seventh detection probe comprises the nucleotide sequence of SEQ ID NO: 9.

Embodiment 48 is the oligonucleotide set of any one of embodiments 42-47, wherein the plurality of stx-specific amplification oligomers comprises first, second, third, fourth, fifth, and sixth oligomers respectively comprising target-hybridizing sequences substantially correspond to nucleotide sequences of: (i) SEQ ID NO: 29; (ii) SEQ ID NO: 5; (iii) SEQ ID NO: 23; (iv) SEQ ID NO: 26; (v) SEQ ID NO: 33; and (vi) SEQ ID NO: 40.

Embodiment 49 is the oligonucleotide set of any one of embodiments 42-47, wherein the plurality of stx-specific amplification oligomers comprises first, second, third, fourth, and fifth oligomers respectively comprising target-hybridizing sequences substantially corresponding to nucleotide sequences of: (i) SEQ ID NO: 14; (ii) SEQ ID NO: 15; (iii) SEQ ID NO: 18; (iv) SEQ ID NO: 30; and (v) SEQ ID NO: 20.

Embodiment 50 is the oligonucleotide set of any one of embodiments 42-47, wherein the plurality of stx-specific amplification oligomers comprises first, second, third, fourth, and fifth oligomers respectively comprising target-hybridizing sequences substantially corresponding to nucleotide sequences of: (i) SEQ ID NO: 21; (ii) SEQ ID NO: 19; (iii) SEQ ID NO: 18; (iv) SEQ ID NO: 30; and (v) SEQ ID NO: 20.

Embodiment 51 is the oligonucleotide set of any one of embodiments 42-47, wherein the plurality of stx-specific amplification oligomers comprises first, second, third, fourth, and fifth oligomers respectively comprising target-hybridizing sequences substantially corresponding to nucleotide sequences of: (i) SEQ ID NO: 31 and; (ii) SEQ ID NO: 27; (iii) SEQ ID NO: 18; (iv) SEQ ID NO: 30; and (v) SEQ ID NO: 20.

Embodiment 52 is the oligonucleotide set of any one of embodiments 42-47, wherein the plurality of stx-specific amplification oligomers comprises first, second, third, fourth, and fifth oligomers respectively comprising target-hybridizing sequences substantially corresponding to nucleotide sequences of: (i) SEQ ID NO: 36; (ii) SEQ ID NO: 27; (iii) SEQ ID NO: 18; (iv) SEQ ID NO: 30; and (v) SEQ ID NO: 20.

Embodiment 53 is the oligonucleotide set of any one of embodiments 42-47, wherein the plurality of stx-specific amplification oligomers comprises first, second, third, fourth, fifth, sixth, and seventh oligomers respectively comprising target-hybridizing sequences substantially corresponding to nucleotide sequences of: (i) SEQ ID NO: 3; (ii) SEQ ID NO: 4; (iii) SEQ ID NO: 18; (iv) SEQ ID NO: 30; (v) SEQ ID NO: 20; (vi) SEQ ID NO: 1; and (vii) SEQ ID NO: 2.

Embodiment 54 is the oligonucleotide set of embodiment 48, wherein the nucleotide sequences of the first, second, third, fourth, fifth, and sixth oligomers respectively comprise or consist of: (i) SEQ ID NO: 29; (ii) SEQ ID NO: 5; (iii) SEQ ID NO: 23; (iv) SEQ ID NO: 26; (v) SEQ ID NO: 33; and (vi) SEQ ID NO: 40.

Embodiment 55 is the oligonucleotide set of embodiment 49, wherein the nucleotide sequences of the first, second, third, fourth, and fifth oligomers respectively comprise or consist of: (i) SEQ ID NO: 14; (ii) SEQ ID NO: 15; (iii) SEQ ID NO: 18; (iv) SEQ ID NO: 30; and (v) SEQ ID NO: 20.

Embodiment 56 is the oligonucleotide set of embodiment 50, wherein the nucleotide sequences of the first, second, third, fourth, and fifth oligomers respectively comprise or consist of: (i) SEQ ID NO: 21; (ii) SEQ ID NO: 19; (iii) SEQ ID NO: 18; (iv) SEQ ID NO: 30; and (v) SEQ ID NO: 20.

Embodiment 57 is the oligonucleotide set of embodiment 51, wherein the nucleotide sequences of the first, second, third, fourth, and fifth oligomers respectively comprise or consist of: (i) SEQ ID NO: 31 and; (ii) SEQ ID NO: 27; (iii) SEQ ID NO: 18; (iv) SEQ ID NO: 30; and (v) SEQ ID NO: 20.

Embodiment 58 is the oligonucleotide set of embodiment 52, wherein the nucleotide sequences of the first, second, third, fourth, and fifth oligomers respectively comprise or consist of: (i) SEQ ID NO: 36; (ii) SEQ ID NO: 27; (iii) SEQ ID NO: 18; (iv) SEQ ID NO: 30; and (v) SEQ ID NO: 20.

Embodiment 59 is the oligonucleotide set of embodiment 53, wherein the nucleotide sequences of the first, second, third, fourth, fifth, sixth, and seventh oligomers respectively comprise or consist of: (i) SEQ ID NO: 3; (ii) SEQ ID NO: 4; (iii) SEQ ID NO: 18; (iv) SEQ ID NO: 30; (v) SEQ ID NO: 20; (vi) SEQ ID NO: 1; and (vii) SEQ ID NO: 2.

Embodiment 60 is the oligonucleotide set of any one of embodiments 42-59, further comprising a plurality of detection probes, wherein the plurality of detection probes comprises a first detection probe comprising a target-hybridizing sequence substantially corresponding to a nucleotide sequence of SEQ ID NO: 28; a second detection probe comprising a target-hybridizing sequence substantially corresponding to a nucleotide sequence of SEQ ID NO: 22; a third detection probe comprising a target-hybridizing sequence substantially corresponding to a nucleotide sequence of SEQ ID NO: 39; a fourth detection probe comprising a target-hybridizing sequence substantially corresponding to a nucleotide sequence of SEQ ID NO: 16; a fifth detection probe comprising a target-hybridizing sequence substantially corresponding to a nucleotide sequence of SEQ ID NO: 42; a sixth detection probe comprising a target-hybridizing sequence substantially corresponding to a nucleotide sequence of SEQ ID NO: 38; and a seventh detection probe comprising a target-hybridizing sequence substantially corresponding to a nucleotide sequence of SEQ ID NO: 25.

Embodiment 61 is the oligonucleotide set of any one of embodiments 42-59, further comprising a plurality of detection probes, wherein the plurality of detection probes comprises a first detection probe comprising a target-hybridizing sequence substantially corresponding to a nucleotide sequence of SEQ ID NO: 17; a second detection probe comprising a target-hybridizing sequence substantially corresponding to a nucleotide sequence of SEQ ID NO: 22; a third detection probe comprising a target-hybridizing sequence substantially corresponding to a nucleotide sequence of SEQ ID NO: 39; a fourth detection probe comprising a target-hybridizing sequence substantially corresponding to a nucleotide sequence of SEQ ID NO: 16; a fifth detection probe comprising a target-hybridizing sequence substantially corresponding to a nucleotide sequence of SEQ ID NO: 42; a sixth detection probe comprising a target-hybridizing sequence substantially corresponding to a nucleotide sequence of SEQ ID NO: 38; and a seventh detection probe comprising a target-hybridizing sequence substantially corresponding to a nucleotide sequence of SEQ ID NO: 25.

Embodiment 62 is the oligonucleotide set of any one of embodiments 42-59, further comprising a plurality of detection probes, wherein the plurality of detection probes comprises a first detection probe having a target-hybridizing sequence comprising or consisting of SEQ ID NO: 28; a second detection probe having a target-hybridizing sequence comprising or consisting of SEQ ID NO: 22; a third detection probe having a target-hybridizing sequence comprising or consisting of SEQ ID NO: 39; a fourth detection probe having a target-hybridizing sequence comprising or consisting of SEQ ID NO: 16; a fifth detection probe having a target-hybridizing sequence comprising or consisting of SEQ ID NO: 42; a sixth detection probe having a target-hybridizing sequence comprising or consisting of SEQ ID NO: 38; and a seventh detection probe having a target-hybridizing sequence comprising or consisting of SEQ ID NO: 25.

Embodiment 63 is the oligonucleotide set of any one of embodiments 42-59, further comprising a plurality of detection probes, wherein the plurality of detection probes comprises a first detection probe having a target-hybridizing sequence comprising or consisting of SEQ ID NO: 17; a second detection probe having a target-hybridizing sequence comprising or consisting of SEQ ID NO: 22; a third detection probe having a target-hybridizing sequence comprising or consisting of SEQ ID NO: 39; a fourth detection probe having a target-hybridizing sequence comprising or consisting of SEQ ID NO: 16; a fifth detection probe having a target-hybridizing sequence comprising or consisting of SEQ ID NO: 42; a sixth detection probe having a target-hybridizing sequence comprising or consisting of SEQ ID NO: 38; and a seventh detection probe having a target-hybridizing sequence comprising or consisting of SEQ ID NO: 25.

Embodiment 64 is the oligonucleotide set of any one of embodiments 62-64, wherein each of the plurality of detection probes is immobilized on a solid support.

Embodiment 65 is the oligonucleotide set of any one of embodiments 62-64, wherein each of the plurality of detection probes is immobilized on a microarray.

Embodiment 66 is the oligonucleotide set of any one of embodiments 62-65, wherein each of the plurality of detection probes comprises a fluorescent label.

Embodiment 67 is the oligonucleotide set of embodiment 66, wherein each of the plurality of detection probes further comprises a non-fluorescent quenching label.

Embodiment 68 is the oligonucleotide set of any one of embodiments 42-65, wherein one or more of the stx-specific amplification oligomers comprises a fluorescent label.

Embodiment 69 is the oligonucleotide set of any one of embodiment 68, wherein two or more of the stx-specific amplification oligomers comprise a fluorescent label so that each amplification product corresponding to a stx target region comprises a fluorescent label incorporated from a stx-specific amplification oligomer.

Embodiment 70 is the oligonucleotide set of embodiment 68 or 69, wherein stx-specific amplification oligomer(s) comprising a sequence substantially corresponding to (i) SEQ ID NOs: 29, 5, 23, and 26; (ii) SEQ ID NOs: 14 and 15; (iii) SEQ ID NOs: 21 and 19; (iv) SEQ ID NOs: 31 and 27; (v) SEQ ID NO: 36; or (vi) SEQ ID NOs: 3, 4, 1, and 2 comprise a fluorescent label.

Embodiment 71 is the oligonucleotide set of embodiment 68 or 69, wherein stx-specific amplification oligomer(s) comprising a sequence substantially corresponding to (i) SEQ ID NOs: 33 and 40; (ii) SEQ ID NOs: 30 and 20; or (iii) SEQ ID NOs: 18, 30, and 20 comprise a fluorescent label.

Embodiment 72 is a kit comprising the oligonucleotide set of any one of embodiments 42-71.

Embodiment 73 is a reaction mixture comprising the oligonucleotide set of any one of embodiments 42-71.

### Definitions

Unless defined otherwise, all technical and scientific terms used herein have the same meaning as commonly understood by one of ordinary skill in the art pertinent to the methods and compositions described. As used herein, the following terms and phrases have the meanings ascribed to them unless specified otherwise.

The terms “a,” “an,” and “the” include plural referents, unless the context clearly indicates otherwise. For example, “a nucleic acid” as used herein is understood to represent one or more nucleic acids. As such, the terms “a” (or “an”), “one or more,” and “at least one” can be used interchangeably herein.

“Sample” refers to any material that may contain or is suspected of containing one or more of bacterial Shiga toxin (stx) variant components, such as nucleic acids or fragments of nucleic acids. A sample may be a complex mixture of components. Samples include “biological samples” which include any tissue or material derived from a living or dead mammal or organism, including, for example, stool, blood, plasma, serum, blood cells, saliva, mucous and cerebrospinal fluid. Samples may also include samples of in vitro cell culture constituents including, for example, conditioned media resulting from the growth of cells and tissues in culture medium. Samples also include food, which includes any material intended or suitable for consumption, including solids, suspensions, emulsions, gels, and liquids (thus including gelatin, milk, soups, beverages, ice-cream, fruit smoothies, emulsified cheese dips, fruit puree, nut butter, processed and/or textured protein, and the like, as well as bread, fruit, vegetables, and meat.). Samples also include water and aqueous solutions. In one step of the methods described herein, a sample is provided that is suspected of containing at least one Shigella or E. coli target nucleic acid. Accordingly, this step excludes the physical step of obtaining the sample from a subject.

“Sample preparation” refers to any steps or method that treats a sample for subsequent amplification and/or detection of stx nucleic acids present in the sample. Samples may be complex mixtures of components (as discussed above) of which the target nucleic acid is a minority component. Sample preparation may include any known method of concentrating components from a larger sample volume, such as by filtration of airborne or waterborne particles from a larger volume sample or by isolation of microbes from a sample by using standard microbiology methods. A sample may be treated chemically, physically or mechanically to disrupt tissue or cell structure to release intracellular nucleic acids into a solution. Samples may be treated to release nucleic acids into a substantially aqueous or organic phase solution containing enzymes, buffers, salts, detergents and the like. Samples may be treated for removal of debris, such as by using filtration, centrifugation or adsorption.

“Nucleic acid” refers to a multimeric compound comprising two or more covalently bonded nucleosides or nucleoside analogs having nitrogenous heterocyclic bases, or base analogs, where the nucleosides are linked together by phosphodiester bonds or other linkages to form a polynucleotide. Nucleic acids include RNA, DNA, or chimeric DNA-RNA polymers, or oligomers (which are synonymous with oligonucleotides), and analogs thereof. Nitrogenous bases may be conventional bases (A, G, C, T, U), analogs thereof (e.g., inosine, 5-methylisocytosine, isoguanine; see, e.g., The Biochemistry of the Nucleic Acids 5-36, Adams et al., ed., 11th ed., 1992; Abraham et al., 2007, BioTechniques 43: 617-24), which include derivatives of purine or pyrimidine bases (e.g., N⁴-methyl deoxyguanosine, deaza- or aza-purines, deaza- or aza-pyrimidines, etc.; U.S. Pat. Nos. 5,378,825, 6,949,367 and International Patent Application Pub. No. WO 93/13121, each incorporated by reference herein). Nucleic acids may include “abasic” residues in which the backbone does not include a nitrogenous base for one or more residues (see, e.g., U.S. Pat. No. 5,585,481, incorporated by reference herein).

A “target nucleic acid” as used herein is a nucleic acid comprising a target region to be amplified. Target nucleic acids may be DNA or RNA as described herein, and may be either single-stranded or double-stranded. In a preferred embodiment, the target nucleic acid is DNA. The target nucleic acid may include other sequences besides the target region, which may not be amplified.

The term “region,” as used herein, refers to a portion of a nucleic acid wherein the portion is smaller than the entire nucleic acid. The term “target region” as used herein refers to the particular nucleotide sequence of the target nucleic acid that is to be amplified and/or detected. The “target region” includes the complexing sequences to which oligomers (e.g., priming oligomers and/or detection probe oligomers) complex during an amplification and/or detection reaction (e.g., PCR). Where the target nucleic acid is originally single-stranded, the term “target region” will also refer to the sequence complementary to the “target region” as present in the target nucleic acid. Where the target nucleic acid is originally double-stranded, the term “target region” refers to both the sense (+) and antisense (−) strands. For example, when the nucleic acid is a segment of a stx gene (e.g., a stx2 gene as represented by any one of SEQ ID NOs: 46-58), the term “target region” may be used to refer to a smaller area of the nucleic acid and/or to a portion of the target nucleic acid to be amplified, wherein the smaller area is targeted by one or more oligomers of the present disclosure. As another non-limiting example, the term “target region” may be used to refer to the sequence to which the target-hybridizing sequence of a probe hybridizes.

“Target-hybridizing sequence” is used herein to refer to the portion of an oligomer that is configured to hybridize with a sequence of a target region. Preferably, a target-hybridizing sequence is configured to specifically hybridize with nucleic acids of a target region. Target-hybridizing sequences may be 100% complementary to the portion of the target region to which they are configured to hybridize, but not necessarily. Target-hybridizing sequences may also include inserted, deleted and/or substituted nucleotide residues relative to a target region. Less than 100% complementarity of a target-hybridizing sequence to a target region may arise, for example, when the target nucleic acid is contained within a plurality of strains within a species. It is understood that other reasons exist for configuring a target-hybridizing sequence to have less than 100% complementarity to a target region.

Oligomer target-hybridizing sequences defined herein by reference to a specific sequence (e.g., by reference to a primer or probe nucleotide sequence) are also understood to include functional complements thereof, unless the context clearly dictates otherwise. Thus, for example, where target-hybridizing sequences of first and second amplification oligomers are defined by reference to specific sequences corresponding, respectively, to sense and/or antisense strands of a target region, it is understood that the amplification oligomer combination may include a functional combination of first and second amplification oligomers having target-hybridizing sequences that are the respective complements of the specific reference regions. Similarly, and again by way of example, where a target-hybridizing sequence for a detection probe oligomer is defined in reference to a specific sequence (e.g., within a target region), it is understood that the detection probe may include a corresponding target-hybridizing sequence that is the complement of the specific sequence within the target region.

The term “configured to” denotes an actual arrangement of the polynucleotide sequence configuration of a referenced oligomer target-hybridizing sequence. For example, amplification oligomers that are configured to generate a specified amplicon from a target region have polynucleotide sequences that hybridize to the target region and can be used in an amplification reaction to generate the amplicon. The term “configured to specifically hybridize to” as used herein means that the target-hybridizing sequence of an amplification oligomer, detection probe, or other oligomer is designed to have a polynucleotide sequence that could target a sequence of the referenced target region. “Specifically hybridize to” means that the oligomer is configured to function in an assay to primarily hybridize to the target so that an accurate detection of target nucleic acid in a sample can be determined; some small level of hybridization to non-target nucleic acids may occur, as is understood in the art. Under stringent hybridization assay conditions, oligomers “preferentially hybridize” to their target regions, or replicates thereof, to form stable oligomer:target hybrids, while at the same time formation of stable oligomer:non-target hybrids is minimized. Thus, an oligomer hybridizes to a target region or replicate thereof to a sufficiently greater extent than to a non-target region, to enable detection of the target region and amplicon thereof. Such an oligomer is not limited to targeting that sequence only, but is rather useful as a composition, in a kit or in a method for targeting a stx target nucleic acid. The oligomer is designed to function as a component of an assay for amplification and detection of one or more stx variants from a sample, and therefore is designed to target one or more stx variants in the presence of other nucleic acids commonly found in testing samples. Appropriate hybridization conditions are well-known in the art for detection probe, amplification, target capture, and other oligomers, and may be predicted based on sequence composition, or can be determined by using routine testing methods (see, e.g., Sambrook et al., Molecular Cloning, A Laboratory Manual, 2^{nd} ed. (Cold Spring Harbor Laboratory Press, Cold Spring Harbor, NY, 1989) at §§ 1.90-1.91, 7.37-7.57, 9.47-9.51 and 11.47-11.57, particularly §§ 9.50-9.51, 11.12-11.13, 11.45-11.47 and 11.55-11.57, incorporated by reference herein in its entirety).

The interchangeable terms “oligomer,” “oligo,” and “oligonucleotide” refer to a nucleic acid having generally less than 1,000 nucleotide (nt) residues, including polymers in a range having a lower limit of about 5 nt residues and an upper limit of about 500 to 900 nt residues. In some embodiments, oligomers are in a size range having a lower limit of about 12 to 15 nt and an upper limit of about 50 to 600 nt, and other embodiments are in a range having a lower limit of about 15 to 20 nt and an upper limit of about 22 to 100 nt. Oligomers may be purified from naturally occurring sources or may be synthesized using any of a variety of well-known enzymatic or chemical methods. The term oligomer does not denote any particular function to the reagent; rather, it is used generically to cover all such reagents described herein. An oligomer may serve various different functions. For example, it may function as a primer if it is specific for and capable of hybridizing to a complementary strand and can further be extended in the presence of a nucleic acid polymerase, and it may function to detect a target nucleic acid if it is capable of hybridizing to the target nucleic acid, or an amplicon thereof, and further provides a detectible moiety. An “amplification oligomer” is an oligomer, at least the 3′-end of which is complementary to a target nucleic acid, and which hybridizes to a target nucleic acid, and participates in a nucleic acid amplification reaction. An example of an amplification oligomer is a “primer” that hybridizes to a target nucleic acid and contains a 3′ OH end that is extended by a polymerase in an amplification process. Size ranges for amplification oligomers include those that are about 10 to about 70 nt long and contain at least about 10 contiguous bases, or even at least 12 contiguous bases that are complementary to a region of the target nucleic acid (or a complementary strand thereof). The contiguous bases are typically at least 80%, at least 90%, at least 95%, or completely complementary to the sequence in a target region, to which the amplification oligomer binds. An amplification oligomer may optionally include modified nucleotides or analogs, or additional nucleotides that participate in an amplification reaction but are not complementary to or contained in the target nucleic acid, or template sequence. It is understood that when referring to ranges for the length of an oligomer, amplicon, or other nucleic acid, that the range is inclusive of all whole numbers (e.g., 15-27 contiguous nucleotides in length includes 15, 16, 17, 18, 19, 20, 21, 22, 23, 24, 25, 26, and 27). It is understood that when referring to percent complementarity, percent identity and the like for an oligomer, amplicon, or other nucleic acid, that range is inclusive of all whole and partial numbers (e.g., 83%-89% includes 83%, 84.75%, 85.6%, 86%, 87%, 87.1%, 89%, etc.).

As used herein, an oligomer “substantially corresponding to” a specified reference nucleic acid sequence means that the oligomer is sufficiently similar to the reference nucleic acid sequence such that the oligomer has similar hybridization properties to the reference nucleic acid sequence in that it would hybridize with the same target nucleic acid region under stringent hybridization conditions. One skilled in the art will understand that “substantially corresponding oligomers” can vary from a reference sequence and still hybridize to the same target nucleic acid region. It is also understood that a first nucleic acid substantially corresponding to a second nucleic acid includes the RNA and DNA thereof and includes the complements thereof, unless the context clearly dictates otherwise. This variation from the nucleic acid may be stated in terms of a percentage of identical bases within the sequence or the percentage of perfectly complementary bases between the probe or primer and its target region. Thus, in certain embodiments, an oligomer “substantially corresponds” to a reference nucleic acid sequence if these percentages of base identity or complementarity are from 100% to about 80%. In preferred embodiments, the percentage is from 100% to about 85%. In more preferred embodiments, this percentage is from 100% to about 90%; in other preferred embodiments, this percentage is from 100% to about 95%. Similarly, a region of a nucleic acid or amplified nucleic acid can be referred to herein as corresponding to a reference nucleic acid sequence. One skilled in the art will understand the various modifications to the hybridization conditions that might be required at various percentages of complementarity to allow hybridization to a specific sequence in a target region without causing an unacceptable level of non-specific hybridization.

“Amplification” refers to any known procedure for obtaining multiple copies of a nucleic acid sequence within a target region or its complement or fragments thereof. The multiple copies may be referred to as amplicons or amplification products. Known amplification methods include both thermal cycling and isothermal amplification methods. Polymerase chain reaction (PCR), replicase-mediated amplification, ligase chain reaction (LCR), strand-displacement amplification (SDA), and transcription-associated amplification (e.g., transcription-mediated amplification (TMA) or NASBA) are non-limiting examples of nucleic acid amplification methods. See, e.g., U.S. Pat. Nos. 4,868,105; 5,124,246; 5,130,238; 5,399,491; 5,437,990; 5,554,516; and 7,374,885; and PCT Pub. Nos. WO 88/01302; WO 88/10315 and WO 95/03430 (TMA); U.S. Pat. No. 4,786,600 (RCA); U.S. Pat. Nos. 5,427,930 and 5,516,663 (LCR); and U.S. Pat. Nos. 5,422,252; 5,547,861; and U.S. Pat. No. 5,648,211 (SDA), each of which is incorporated herein by reference in its entirety. See also, e.g., Compton, Nature 350:91-92, 1991; Malek et al., Methods Mol. Biol. 28:253-260, 1994 (NASBA), each of which is incorporated by reference herein in its entirety. PCR is the preferred amplification method, and is well-known in the art. Briefly, PCR amplification uses a DNA polymerase, pairs of primers, and thermal cycling to synthesize multiple copies of two complementary strands from dsDNA or from a cDNA (see, e.g., U.S. Pat. Nos. 4,683,195, 4,683,202, and 4,800,159, each of which is incorporated herein by reference in its entirety).

As used herein, the term “real-time amplification” refers to amplification of target nucleic acid that is monitored by real-time detection means. Real-time PCR amplification includes a method and reagents for performing what is commonly referred to as Taqman® PCR (see, e.g., Holland et al., Proc. Natl. Acad. Sci. USA 88:7276-7280, 1991; and Livak et al., U.S. Pat. No. 6,030,787, each of which is incorporated herein by reference in its entirety).

The term “amplicon” or the term “amplification product” as used herein refers to the nucleic acid molecule generated during an amplification procedure that is complementary or homologous to a sequence contained within the target region. These terms can be used to refer to a single-stranded amplification product, a double-stranded amplification product, or one of the strands of a double-stranded amplification product.

A “non-target-specific sequence,” as is used herein refers to a region of an oligomer sequence, wherein said region does not stably hybridize with a sequence in a target region under standard hybridization conditions. Detection probe oligomers with non-target-specific sequences include, but are not limited to, molecular beacons and molecular torches. Amplification oligomers with non-target-specific sequences include, but are not limited to, hairpin primers and scorpion primers. An amplicon that is generated by an amplification oligomer with non-target-specific sequences will comprise the non-target-specific sequence and the complement thereof.

A “detection probe,” “detection oligomer,” and “detection probe oligomer” are used interchangeably to refer to a nucleic acid oligomer that hybridizes specifically to a target region in a nucleic acid, or in an amplified nucleic acid, under conditions that promote hybridization to allow detection of the nucleic acid or amplified nucleic acid. Probe lengths are preferably in the range from 10 nucleobases to 100 nucleobases, inclusive of all whole numbers therein. Detection may either be direct (e.g., a probe hybridized directly to a sequence in its target region) or indirect (e.g., a probe linked to its target via an intermediate molecular structure). Detection probes may be DNA, RNA, analogs thereof or combinations thereof and they may be labeled or unlabeled. Detection probes may further include alternative backbone linkages. For example, detection probes may comprise a 2′-O-methyl residue, which can result in a higher signal being obtained. A detection probe's “target region” generally refers to a smaller nucleic acid sequence region within a larger nucleic acid sequence (e.g., a target nucleic acid) that hybridizes specifically to at least a portion of a probe oligomer by standard base pairing. A detection probe may comprise target-hybridizing sequences and, optionally, other sequences that contribute to the three-dimensional conformation of the probe (see, e.g., U.S. Pat. Nos. 5,118,801; 5,312,728; 6,849,412; 6,835,542; 6,534,274; and 6,361,945; and US Patent Application Pub. No. 20060068417; each incorporated by reference herein). In general, the term “TaqMan® probe” refers to a dual labelled detection probe oligomer that contains a fluorescent dye, typically but not necessarily on the 5′ base, and a non-fluorescent quenching label or dye (quencher), typically but not necessarily on the 3′ base. When irradiated, the excited fluorescent dye transfers energy to the nearby quenching dye molecule rather than fluorescing, resulting in a non-fluorescent substrate. During amplification, the exonuclease activity of the DNA polymerase cleaves the TaqMan® probe to separate the fluorophore from the quencher, thereby allowing an unquenched signal to be emitted from the fluorophore as an indicator of amplification.

By “stable” or “stable for detection” is meant that the temperature of a reaction mixture is at least 2° C. below the melting temperature of a nucleic acid duplex.

As used herein, a “label” refers to a moiety or compound joined directly or indirectly to an oligomer, such as a probe or amplification oligomer, that is detected or leads to a detectable signal. Direct labeling can occur through bonds or interactions that link the label to the oligomer, including covalent bonds or non-covalent interactions, e.g., hydrogen bonds, hydrophobic and ionic interactions, or formation of chelates or coordination complexes. Indirect labeling can occur through use of a bridging moiety or “linker” such as a binding pair member, an antibody or additional oligomer, which is either directly or indirectly labeled, and which may amplify the detectable signal. Labels include any detectable moiety, such as a radionuclide, ligand (e.g., biotin, avidin), enzyme or enzyme substrate, reactive group, or chromophore (e.g., dye, particle, or bead that imparts detectable color), luminescent compound (e.g., bioluminescent, phosphorescent, or chemiluminescent labels), or fluorophore. Labels may be detectable in a homogeneous assay in which bound labeled oligomer in a mixture exhibits a detectable change different from that of an unbound labeled oligomer, e.g., instability or differential degradation properties. A “homogeneous detectable label” can be detected without physically removing bound from unbound forms of the label or labeled oligomer (see, e.g., U.S. Pat. Nos. 5,118,801; 5,283,174; 5,312,728; 5,656,207; and 5,658,737; each incorporated by reference herein in its entirety). Common labels used for TaqMan® probes include a fluorophore and a quencher. Exemplary fluorophores include FAM, SYBR® Green, ATTO fluorescent labels, VIC, JOE, NED, Cy3, ROX, Texas Red and Cy5 dyes (all well-known in the art and readily available from numerous commercial sources). Exemplary quenchers include BBQ, ATTO quenchers, BHQ, TAMRA and DABCYL (all well-known in the art and readily available from numerous commercial sources). Synthesis and methods of attaching labels to nucleic acids and detecting labels are well known (see for example, Sambrook et al., Molecular Cloning, A Laboratory Manual, 2nd ed. (Cold Spring Harbor Laboratory Press, Cold Spring Harbor, NY, 1989), Chapter 10; U.S. Pat. Nos. 5,658,737, 5,656,207, 5,547,842, 5,283,174, and 4,581,333, each of which is incorporated herein by reference in entirety). More than one label, and more than one type of label, may be present on a particular oligomer, or detection may use a mixture of oligomers in which each oligomer is labelled with a compound that produces a different detectable signal (see, e.g., U.S. Pat. Nos. 6,180,340 and 6,350,579, each of which is incorporated herein by reference in its entirety).

As used herein, an “immobilized oligomer” or “immobilized probe” refers to an oligomer attached to a support, directly or indirectly. An immobilized probe joined to a support facilitates immobilization and/or separation of a target, e.g., from or relative to other material in a sample that does not specifically bind to the capture oligomer. Supports may include known materials, such as matrices, microarrays, and particles free in solution, which may be made of nitrocellulose, nylon, glass, one or more polymers, or other compositions, of which one embodiment is magnetically attractable particles. The linkage or interaction between the probe and support is stable during hybridization conditions.

“Separating,” “purifying,” or “isolating” means that one or more components of a sample are removed or separated from other sample components. Sample components include target nucleic acids usually in a generally aqueous solution phase, which may also include cellular fragments, proteins, carbohydrates, lipids, and other nucleic acids. Separating or purifying removes at least 70%, or at least 80%, or at least 95% of the target nucleic acid from other sample components. Sample preparation may include use of an immobilized oligomer that selectively or non-specifically captures a target nucleic acid and separates it from other sample components (e.g., as described in U.S. Pat. No. 6,110,678 and International Patent Application Pub. No. WO 2008/016988, each incorporated by reference herein in its entirety).

The term “complementary” means that the nucleotide sequences of similar regions of two single-stranded nucleic acids, or to different regions of the same single-stranded nucleic acid have a nucleotide base composition that allow the single-stranded regions to hybridize together in a stable double-stranded hydrogen-bonded region under stringent hybridization or amplification conditions. Sequences that hybridize to each other may be completely complementary or partially complementary to the intended target region by standard nucleic acid base pairing (e.g., G:C, A:T or A:U pairing). By “sufficiently complementary” is meant a contiguous sequence that is capable of hybridizing to another sequence by hydrogen bonding between a series of complementary bases, which may be complementary at each position in the sequence by standard base pairing or may contain one or more residues, including abasic residues, that are not complementary. Sufficiently complementary contiguous sequences typically are at least 80%, or at least 90%, complementary to a sequence to which an oligomer is intended to specifically hybridize. Sequences that are “sufficiently complementary” allow stable hybridization of a nucleic acid oligomer with its target region under appropriate hybridization conditions, even if the sequences are not completely complementary. When a contiguous sequence of nucleotides of one single-stranded region is able to form a series of “canonical” hydrogen-bonded base pairs with an analogous sequence of nucleotides of the other single-stranded region, such that A is paired with U or T and C is paired with G, the nucleotides sequences are “completely” complementary (see, e.g., Sambrook et al., Molecular Cloning, A Laboratory Manual, 2^{nd} ed. (Cold Spring Harbor Laboratory Press, Cold Spring Harbor, NY, 1989) at §§ 1.90-1.91, 7.37-7.57, 9.47-9.51 and 11.47-11.57, particularly §§ 9.50-9.51, 11.12-11.13, 11.45-11.47 and 11.55-11.57, incorporated by reference herein). It is understood that ranges for percent identity are inclusive of all whole and partial numbers (e.g., at least 90% includes 90%, 91%, 93.5%, 97.687%, 99%, 100% and etc.).

By “nucleic acid hybrid,” “hybrid,” or “duplex” is meant a nucleic acid structure containing a double-stranded, hydrogen-bonded region wherein each strand is at least sufficiently complementary to the other, and wherein the region is sufficiently stable under stringent hybridization conditions to be detected by means including, but not limited to, chemiluminescent or fluorescent light detection, autoradiography, or gel electrophoresis. Such hybrids may comprise RNA:RNA, RNA:DNA, or DNA:DNA duplex molecules. The term “specificity,” in the context of an amplification and/or detection system, is used herein to refer to the characteristic of the system which describes its ability to distinguish between target and non-target regions dependent on sequence and assay conditions. In terms of nucleic acid amplification, specificity generally refers to the ratio of the number of specific amplicons produced to the number of side-products (e.g., the signal-to-noise ratio). In terms of detection, specificity generally refers to the ratio of signal produced from target nucleic acids to signal produced from non-target nucleic acids.

The term “sensitivity” is used herein to refer to the precision with which a nucleic acid amplification reaction can be detected or quantitated. The sensitivity of an amplification reaction is generally a measure of the smallest copy number of the target nucleic acid that can be reliably detected in the amplification system, and will depend, for example, on the detection assay being employed, and the specificity of the amplification reaction, e.g., the ratio of specific amplicons to side-products.

### DETAILED DESCRIPTION

The present disclosure provides compositions, kits, and methods for amplifying and/or detecting shiga toxin (stx) nucleic acid from a sample. For example, the presently disclosed compositions, kits, and methods can be used for amplifying and/or detecting stx2 nucleic acid from a sample The compositions, kits and methods provide oligomers, each oligomer recognizing a sequence within a stx target region or its complementary sequence. The oligomers may serve as amplification oligomers and/or detection probes for amplification and/or detection of stx target nucleic acid. An amplification oligomer is configured to specifically hybridize to a stx target region within a target nucleic acid. At least two amplification oligomers flanking a target region within the target nucleic acid are utilized in an in vitro nucleic acid amplification reaction to generate an amplicon therefrom. Exemplary in vitro amplification reactions include, for example, PCR (e.g., Tagman@PCR) and transcription-associated amplification (e.g., TMA or NASBA). A detection probe, configured to specifically hybridize to a target region flanked by at least two amplification oligomers, may be utilized to hybridize specifically to at least a portion of an amplification product, either after completion of or during the amplification process. Methods of the present disclosure may further use an oligomer that serves as a capture probe for processing a sample by capturing a stx target nucleic acid and separating it from other sample components (see, e.g., U.S. Pat. Nos. 6,110,678, 6,280,952, and 6,534,273, each of which is incorporated by reference herein in its entirety). In any of the embodiments described herein involving an stx target nucleic acid, the stx target nucleic acid may be an stx2 target nucleic acid.

In certain embodiments, oligomers and methods of the present disclosure are useful for amplifying and detecting target regions of stx nucleic acids from bacteria present in a sample in a relatively short time so that diagnosis can be made quickly and so that effective treatment can be initiated to limit the spread of the bacteria. Thus, in some embodiments, the present disclosure responds to a need for rapid, sensitive, and specific testing of clinical samples that may contain stx-producing bacteria.

### I. Amplification and Detection Oligomers

Detection probe oligomer sequences as disclosed herein may be used as amplification oligomers, and amplification oligomer sequences as disclosed herein may be used as detection probes. The same is true for the disclosed probe hybridization regions and amplification oligomer hybridization regions of a given target gene. Thus, the probe hybridization regions disclosed herein may be used as amplification oligomer hybridization regions. Likewise, amplification oligomer hybridization regions disclosed herein may be used as probe hybridization regions. Oligomer sequences are disclosed in the Sequence Table (Table 6) below.

### A. Oligomers for Amplification of Nucleic Acids

Oligomers for amplifying a stx target typically comprise at least two amplification oligomers. Some embodiments of the disclosure may utilize three, four, five, six, seven, or even eight or ten or more amplification oligomers in, for example, multiplex amplification assays. Thus, by way of example, oligomers for amplifying a stx target gene may comprise one, two, three, four, or five or more forward amplification primers and one, two, three, four, or five or more reverse amplification primers. In one embodiment, at least two amplification oligomers are used in order to generate an amplicon that can be subsequently detected, where the at least two amplification oligomers are configured to specifically hybridize to a region within a target nucleic acid selected from a target nucleic corresponding to a stx gene. Suitably, the amplicon is detectable using a detection probe. Typically, the amplicon is from 50 to 300 nucleotides in length (e.g., 50 to 250 nucleotides in length or 90 to 250 nucleotides in length), including all whole numbers between 50 and 300 that are not explicitly listed here. In certain embodiments, a set of oligomers includes amplification oligomers selected from the oligomers above for amplifying two or more variants (e.g., three or four) of a stx target nucleic acid region. In some embodiments, the stx target is an stx2a, stx2b, stx2c, stx2d, stx2e, stx2f stx2g, stx2h, stx2i, stx2j, stx2k, stx2l, and/or stx2m, e.g., from Escherichia coli.

In certain embodiments, at least two amplification oligomers are used in order to generate an amplicon that can be subsequently detected, where the at least two amplification oligomers are configured to specifically hybridize to a target nucleic acid region selected from a region within a nucleic acid sequence corresponding to SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, and/or SEQ ID NO: 58.

In particular embodiments of the present disclosure, the at least two amplification oligomers for amplifying a stx target nucleic acid comprise (i) a first amplification oligomer that includes a target-hybridizing sequence substantially corresponding to, comprising, or consisting of a forward primer oligomer sequence as shown in Table 6, infra, and (ii) a second amplification oligomer that includes a target-hybridizing sequence substantially corresponding to, comprising, or consisting of a reverse primer oligomer sequence, also shown in Table 6, where the first and second amplification oligomers correspond to the same target nucleic acid, and where the target-hybridizing sequences are selected such that, for any oligomer pair, an antisense sequence is situated downstream of a sense sequence (e.g., the first and second amplification oligomers are situated such that they flank a target region to be amplified). In specific variations, the first and/or second amplification oligomer—or the first and/or second target-hybridizing sequence of a first and/or second amplification oligomer—comprises or consists of an oligomer sequence selected from the oligomer sequences shown in Table 6. Although these sequences are shown as DNA sequences, equivalent RNA or equivalent RNA/DNA chimeric sequences can be readily derived by the person skilled in the art and are to be considered as falling within the definition of “oligomer,” “amplification oligomer,” or “primer.” In addition, complementary sequences of DNA and RNA and reverse complementary sequences can be readily derived by the skilled person. It is therefore to be understood that a description of any individual sequence of DNA, for example, encompasses its complement, its reverse complement, and equivalent RNA or RNA/DNA chimeric sequences.

In some embodiments, stx-specific amplification oligomers comprise target-hybridizing sequences substantially corresponding to, or consisting of, the nucleotide sequences of: (i) SEQ ID NO: 29 and SEQ ID NO: 33; (ii) SEQ ID NO: 5 and SEQ ID NO: 33; (iii) SEQ ID NO: 23 and SEQ ID NO: 33; (iv) SEQ ID NO: 26 and SEQ ID NO: 33; (v) SEQ ID NO: 29 and SEQ ID NO: 40; (vi) SEQ ID NO: 5 and SEQ ID NO: 40; (vii) SEQ ID NO: 23 and SEQ ID NO: 40; and/or (viii) SEQ ID NO: 26 and SEQ ID NO: 40. In some embodiments, stx-specific amplification oligomers comprise target-hybridizing sequences substantially corresponding to, or consisting of, the nucleotide sequences of: (i) SEQ ID NO: 14 and 18; (ii) SEQ ID NO: 15 and 18; (iii) SEQ ID NO: 14 and 30; (iv) SEQ ID NO: 15 and 30; (v) SEQ ID NO: 14 and 20; and/or (vi) SEQ ID NO: 15 and 20. In some embodiments, stx-specific amplification oligomers comprise target-hybridizing sequences substantially corresponding to, or consisting of, the nucleotide sequences of: (i) SEQ ID NO: 21 and SEQ ID NO: 18; (ii) SEQ ID NO: 21 and SEQ ID NO: 30; (iii) SEQ ID NO: 21 and SEQ ID NO: 20 (iv) SEQ ID NO: 19 and SEQ ID NO: 18; (v) SEQ ID NO: 19 and SEQ ID NO: 30; and/or (vi) SEQ ID NO: 19 and SEQ ID NO: 20. In some embodiments, stx-specific amplification oligomers comprise target-hybridizing sequences substantially corresponding to, or consisting of, the nucleotide sequences of: (i) SEQ ID NO: 31 and SEQ ID NO: 18; (ii) SEQ ID NO: 31 and SEQ ID NO: 30; (iii) SEQ ID NO: 31 and SEQ ID NO: 20; (iv) SEQ ID NO: 27 and SEQ ID NO: 18; (v) SEQ ID NO: 27 and SEQ ID NO: 30; and/or (vi) SEQ ID NO: 27 and SEQ ID NO: 20. In some embodiments, stx-specific amplification oligomers comprise target-hybridizing sequences substantially corresponding to, or consisting of, the nucleotide sequences of: (i) SEQ ID NO: 36 and SEQ ID NO: 18; (ii) SEQ ID NO: 36 and SEQ ID NO: 30; (iii) SEQ ID NO: 36 and SEQ ID NO: 20; (iv) SEQ ID NO: 27 and SEQ ID NO: 18; (v) SEQ ID NO: 27 and SEQ ID NO: 30; and/or (vi) SEQ ID NO: 27 and SEQ ID NO: 20. In some embodiments, stx-specific amplification oligomers comprise target-hybridizing sequences substantially corresponding to, or consisting of, the nucleotide sequences of: (i) SEQ ID NO: 3 and SEQ ID NO: 18; (ii) SEQ ID NO: 3 and SEQ ID NO: 30; (iii) SEQ ID NO: 3 and SEQ ID NO: 20; (iv) SEQ ID NO: 4 and SEQ ID NO: 18; (v) SEQ ID NO: 4 and SEQ ID NO: 30; (vi) SEQ ID NO: 4 and SEQ ID NO: 20; (vii) SEQ ID NO: 1 and SEQ ID NO: 18; (viii) SEQ ID NO: 1 and SEQ ID NO: 30; (ix) SEQ ID NO: 1 and SEQ ID NO: 20; (x) SEQ ID NO: 2 and SEQ ID NO: 18; (xi) SEQ ID NO: 2 and SEQ ID NO: 30; and/or (xii) SEQ ID NO: 2 and SEQ ID NO: 20. In some embodiments, stx-specific amplification oligomers comprise target-hybridizing sequences substantially corresponding to, or consisting of, the nucleotide sequences of: (i) SEQ ID NO: 37 and SEQ ID NO: 24; (ii) SEQ ID NO: 37 and SEQ ID NO: 24; (iii) SEQ ID NO: 6 and SEQ ID NO: 41; and/or (iv) SEQ ID NO: 6 and SEQ ID NO: 41.

In some embodiments, a plurality of stx-specific amplification oligomers comprises target-hybridizing sequences substantially corresponding to, or consisting of nucleotide sequences of (i) SEQ ID NO: 29; (ii) SEQ ID NO: 5; (iii) SEQ ID NO: 23; (iv) SEQ ID NO: 26; (v) SEQ ID NO: 33; and (vi) SEQ ID NO: 40. In some embodiments, a plurality of stx-specific amplification oligomers comprises target-hybridizing sequences substantially corresponding to, or consisting of nucleotide sequences of (i) SEQ ID NO: 14; (ii) SEQ ID NO: 15; (iii) SEQ ID NO: 18; (iv) SEQ ID NO: 30; and (v) SEQ ID NO: 20. In some embodiments, a plurality of stx-specific amplification oligomers comprises target-hybridizing sequences substantially corresponding to, or consisting of nucleotide sequences of (i) SEQ ID NO: 21; (ii) SEQ ID NO: 19; (iii) SEQ ID NO: 18; (iv) SEQ ID NO: 30; and (v) SEQ ID NO: 20. In some embodiments, a plurality of stx-specific amplification oligomers comprises target-hybridizing sequences substantially corresponding to, or consisting of nucleotide sequences of (i) SEQ ID NO: 31 and; (ii) SEQ ID NO: 27; (iii) SEQ ID NO: 18; (iv) SEQ ID NO: 30; and (v) SEQ ID NO: 20. In some embodiments, a plurality of stx-specific amplification oligomers comprises target-hybridizing sequences substantially corresponding to, or consisting of nucleotide sequences of (i) SEQ ID NO: 36; (ii) SEQ ID NO: 27; (iii) SEQ ID NO: 18; (iv) SEQ ID NO: 30; and (v) SEQ ID NO: 20. In some embodiments, a plurality of stx-specific amplification oligomers comprises target-hybridizing sequences substantially corresponding to, or consisting of nucleotide sequences of (i) SEQ ID NO: 3; (ii) SEQ ID NO: 4; (iii) SEQ ID NO: 18; (iv) SEQ ID NO: 30; (v) SEQ ID NO: 20; (vi) SEQ ID NO: 1; and (vii) SEQ ID NO: 2.

In some embodiments, a plurality of stx-specific amplification oligomers further comprises target-hybridizing sequences substantially corresponding to, or consisting of nucleotide sequences of (i) SEQ ID NO: 37; (ii) SEQ ID NO: 6; (iii) SEQ ID NO: 24; and (iv) SEQ ID NO: 41.

In some embodiments, a plurality of stx-specific amplification oligomers comprises (i) at least one oligomer comprising a nucleic acid sequence substantially corresponding to, or consisting of a nucleotide sequence of SEQ ID NOs: 5, 6, 23, 26, 29, and/or 37; and (ii) an oligomer comprising a nucleic acid sequence substantially corresponding to, or consisting of the nucleotide sequence of SEQ ID NO: 43. In some embodiments, N is an adenine, and/or R is a guanine.

In some embodiments, a plurality of stx-specific amplification oligomers comprises (i) one or more oligomers comprising nucleic acid sequences substantially corresponding to, or consisting of nucleotide sequences of SEQ ID NOs: 18, 20, and/or 30; and (ii) one or more oligomers comprising nucleic acid sequences substantially corresponding to, or consisting of nucleotide sequences of SEQ ID NOs: 1, 2, 3, 4, 14, 15, 19, 21, 26, 27, and 31.

In some embodiments, a plurality of stx-specific amplification oligomers comprises (A) two oligomers respectively comprising nucleic acid sequences substantially corresponding to, or consisting of SEQ ID NOs: 14 and 15; (B) two oligomers respectively comprising nucleic acid sequences substantially corresponding to, or consisting of SEQ ID NOs: 19 and 21; (C) two oligomers respectively comprising nucleic acid sequences substantially corresponding to, or consisting of SEQ ID NOs: 27 and 31; (D) two oligomers respectively comprising nucleic acid sequences substantially corresponding to, or consisting of SEQ ID NOs: 26 and 31; or (E) four oligomers respectively comprising nucleic acid sequences substantially corresponding to, or consisting of SEQ ID NOs: 1, 2, 3, and 4.

In some embodiments, a plurality of stx-specific amplification oligomers comprises (i) one or more oligomers comprising nucleic acid sequences substantially corresponding to, or consisting of nucleotide sequences of SEQ ID NOs: 18, 20, and/or 30; and (ii) one or more oligomers comprising nucleic acid sequences each individual selected from the group consisting of (A) nucleic acid sequences that are from 18 to 22 contiguous nucleotides of SEQ ID NO: 44, and (B) nucleic acid sequences that are from 18 to 20 contiguous nucleotides of SEQ ID NO: 44. In some embodiments, a plurality of stx-specific amplification oligomers comprises (A) two oligomers respectively comprising nucleic acid sequences substantially corresponding to, or consisting of: SEQ ID NOs: 27 and 31; (B) two oligomers respectively comprising nucleic acid sequences substantially corresponding to, or consisting of SEQ ID NOs: 26 and 31; (C) two oligomers respectively comprising nucleic acid sequences substantially corresponding to, or consisting of SEQ ID NOs: 1 and 2; (D) two oligomers respectively comprising nucleic acid sequences substantially corresponding to, or consisting of SEQ ID NOs: 3 and 4; or (E) four oligomers respectively comprising nucleic acid sequences substantially corresponding to, or consisting of SEQ ID NOs: 1, 2, 3, and 4.

### B. Probes for Detection of Nucleic Acids

Methods for detecting a stx target nucleic acid optionally include a detecting step that uses at least one probe that specifically hybridizes to a stx gene amplification product (RNA or DNA amplicon, preferably DNA amplicon). Accordingly, in certain embodiments, a detection probe of the present disclosure is configured to specifically hybridize to a region within a stx target nucleic acid. In certain embodiments, a set of oligomers for detection of a stx includes one, two or more detection probes, where the probes are for detecting one, two or more (e.g., three or four) of a stx target nucleic acid region.

In certain embodiments, a detection probe is configured to specifically hybridize to a target nucleic acid region selected from a region within a nucleic acid sequence corresponding to SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, and/or SEQ ID NO: 58.

In particular embodiments, a detection probe as above—configured to specifically hybridize to a target nucleic acid region selected from a region within a nucleic acid sequence corresponding to SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, and/or SEQ ID NO: 58—includes a target-hybridizing sequence substantially corresponding to, comprising, or consisting of a probe oligomer sequence as shown in Table 6, infra. In specific variations, the detection probe—or the target-hybridizing sequence of a detection probe—comprises or consists of a probe oligomer sequence selected from the oligomer sequences shown in Table 6. Although these sequences are shown as DNA sequences, equivalent RNA or RNA/DNA chimeric sequences can be readily derived by the person skilled in the art and are to be considered as falling within the definition of “oligomer” or “detection probe.” In addition, complementary sequences of DNA and RNA and reverse complementary sequences can be readily derived by the skilled person. It is therefore to be understood that a description of any individual sequence of DNA, for example, encompasses its complement, its reverse complement, and equivalent RNA or RNA/DNA chimeric sequences.

In some embodiments, detection probes comprise target-hybridizing sequences substantially corresponding to, or consisting of, the nucleotide sequences of SEQ ID NO: 28 and/or SEQ ID NO: 17. In some embodiments, detection probes comprise target-hybridizing sequences substantially corresponding to, or consisting of, the nucleotide sequences of SEQ ID NO: 22; SEQ ID NO: 39; SEQ ID NO: 16; SEQ ID NO: 42; SEQ ID NO: 25; SEQ ID NO: 38; SEQ ID NO: 8; SEQ ID NO: 12; SEQ ID NO: 7; SEQ ID NO: 13; SEQ ID NO: 11; SEQ ID NO: 9; and/or SEQ ID NO: 10.

In some embodiments, a plurality of detection probes comprises target-hybridizing sequences substantially corresponding to, or consisting of, the nucleotide sequences of SEQ ID NO: 28; SEQ ID NO: 22; SEQ ID NO: 39; SEQ ID NO: 16; SEQ ID NO: 42; SEQ ID NO:38; and SEQ ID NO: 25. In some embodiments, a plurality of detection probes comprises target-hybridizing sequences substantially corresponding to, or consisting of, the nucleotide sequences of SEQ ID NO: 17; SEQ ID NO: 22; SEQ ID NO: 39; SEQ ID NO: 16; SEQ ID NO: 42; SEQ ID NO: 38; SEQ ID NO: 25.

C. Oligomers for Amplification and Detection of Nucleic acids

In some embodiments, oligomers for amplifying and detecting a stx target typically comprise at least two amplification oligomers and at least one detection probe. Some embodiments may utilize four, five, six, seven, eight or more amplification oligomers and two, three, four, five or even six or more detection probes. Thus, by way of example, oligomers for amplifying and detecting a stx target may comprise two or three or more forward amplification oligomers (e.g., primers) together with two or three or more reverse amplification primers (e.g., primers) together with two, three, four, five or even six or more detection probes.

In some embodiments for determining the presence of absence of a stx target in a sample, oligomers for amplifying and detecting a stx target comprise at least two stx-specific amplification oligomers for amplifying a target region of the stx target nucleic acid, wherein the at least two stx-specific amplification oligomers comprise or consist of target hybridizing sequences substantially corresponding to, or consisting of, nucleotide sequences of (i) SEQ ID NO: 29 and SEQ ID NO: 33; (ii) SEQ ID NO: 5 and SEQ ID NO: 33; (iii) SEQ ID NO: 23 and SEQ ID NO: 33; (iv) SEQ ID NO: 26 and SEQ ID NO: 33; (v) SEQ ID NO: 29 and SEQ ID NO: 40; (vi) SEQ ID NO: 5 and SEQ ID NO: 40; (vii) SEQ ID NO: 23 and SEQ ID NO: 40; and/or (viii) SEQ ID NO: 26 and SEQ ID NO: 40. In some embodiments, the at least two stx-specific amplification oligomers comprise or consist of target hybridizing sequences substantially corresponding to, or consisting of, nucleotide sequences of: (i) SEQ ID NO: 14 and 18; (ii) SEQ ID NO: 15 and 18; (iii) SEQ ID NO: 14 and 30; (iv) SEQ ID NO: 15 and 30; (v) SEQ ID NO: 14 and 20; and/or (vi) SEQ ID NO: 15 and 20. In some embodiments, the at least two stx-specific amplification oligomers comprise or consist of target hybridizing sequences substantially corresponding to, or consisting of, nucleotide sequences of: (i) SEQ ID NO: 21 and SEQ ID NO: 18; (ii) SEQ ID NO: 21 and SEQ ID NO: 30; (iii) SEQ ID NO: 21 and SEQ ID NO: 20 (iv) SEQ ID NO: 19 and SEQ ID NO: 18; (v) SEQ ID NO: 19 and SEQ ID NO: 30; and/or (vi) SEQ ID NO: 19 and SEQ ID NO: 20. In some embodiments, the at least two stx-specific amplification oligomers comprise or consist of target hybridizing sequences substantially corresponding to, or consisting of, nucleotide sequences of: (i) SEQ ID NO: 31 and SEQ ID NO: 18; (ii) SEQ ID NO: 31 and SEQ ID NO: 30; (iii) SEQ ID NO: 31 and SEQ ID NO: 20; (iv) SEQ ID NO: 27 and SEQ ID NO: 18; (v) SEQ ID NO: 27 and SEQ ID NO: 30; and/or (vi) SEQ ID NO: 27 and SEQ ID NO: 20. In some embodiments, the at least two stx-specific amplification oligomers comprise or consist of target hybridizing sequences substantially corresponding to, or consisting of, nucleotide sequences of: (i) SEQ ID NO: 36 and SEQ ID NO: 18; (ii) SEQ ID NO: 36 and SEQ ID NO: 30; (iii) SEQ ID NO: 36 and SEQ ID NO: 20; (iv) SEQ ID NO: 27 and SEQ ID NO: 18; (v) SEQ ID NO: 27 and SEQ ID NO: 30; and/or (vi) SEQ ID NO: 27 and SEQ ID NO: 20. In some embodiments, the at least two stx-specific amplification oligomers comprise or consist of target hybridizing sequences substantially corresponding to, or consisting of, nucleotide sequences of: (i) SEQ ID NO: 3 and SEQ ID NO: 18; (ii) SEQ ID NO: 3 and SEQ ID NO: 30; (iii) SEQ ID NO: 3 and SEQ ID NO: 20; (iv) SEQ ID NO: 4 and SEQ ID NO: 18; (v) SEQ ID NO: 4 and SEQ ID NO: 30; (vi) SEQ ID NO: 4 and SEQ ID NO: 20; (vii) SEQ ID NO: 1 and SEQ ID NO: 18; (viii) SEQ ID NO: 1 and SEQ ID NO: 30; (ix) SEQ ID NO: 1 and SEQ ID NO: 20; (x) SEQ ID NO: 2 and SEQ ID NO: 18; (xi) SEQ ID NO: 2 and SEQ ID NO: 30; and/or (xii) SEQ ID NO: 2 and SEQ ID NO: 20. In some embodiments, the at least two stx-specific amplification oligomers comprise or consist of target hybridizing sequences substantially corresponding to, or consisting of, nucleotide sequences of: (i) SEQ ID NO: 37 and SEQ ID NO: 24; (ii) SEQ ID NO: 37 and SEQ ID NO: 24; (iii) SEQ ID NO: 6 and SEQ ID NO: 41; and/or (iv) SEQ ID NO: 6 and SEQ ID NO: 41.

The oligomers for amplifying and detecting a stx target may further include a detection probe specific for a stx target region flanked by the first and second amplification oligomers. In some embodiments, the detection probe comprises or consists of a target hybridizing sequence substantially corresponding to, or consisting of, the nucleotide sequence of SEQ ID NO: 22; SEQ ID NO: 39; SEQ ID NO: 16; SEQ ID NO: 42; SEQ ID NO: 25; SEQ ID NO: 38; SEQ ID NO: 8; SEQ ID NO: 12; SEQ ID NO: 7; SEQ ID NO: 13; SEQ ID NO: 11; SEQ ID NO: 9; and/or SEQ ID NO: 10.

### II. Assays

Assays for detection of a stx target nucleic acid may include an internal control (IC) nucleic acid that is amplified and detected by using IC-specific primers and probe in the same reaction mixtures used for amplification and detection of a region of one or more stx variant target nucleic acids. Amplification and detection of the IC-specific sequence demonstrates that assay reagents and conditions were properly used even when a signal specific for stx is not detected for a tested sample (e.g., negative samples). The IC may be used as an internal calibrator for the assay that provides a quantitative result. The IC may be a randomized sequence derived from a naturally occurring source bacterium that does not harbor a stx target nucleic acid.

### A. Multiplex Assays

In certain embodiments, a combination of oligomers is provided for amplification and/or detection of at least two stx variants. Such oligomers are particularly useful in a multiplex assay for determining the presence or absence of at least two of stx variants in a sample. In some embodiments, the oligomers include at least two stx-specific amplification oligomers as described above. In some embodiments, the oligomers further include, for each stx variant target region flanked by the at least two amplification oligomers, at least one corresponding detection probe as described above.

### III. Detection Labels

In some embodiments, a detection probe in accordance with the present disclosure further includes a label. In other embodiments, an amplification oligomer comprises a label.

Particularly suitable labels include compounds that emit a detectable light signal, e.g., fluorophores or luminescent (e.g., chemiluminescent) compounds that can be detected in a homogeneous mixture. More than one label, and more than one type of label, may be present on a particular probe, or detection may rely on using a mixture of probes in which each probe is labeled with a compound that produces a detectable signal (see, e.g., U.S. Pat. Nos. 6,180,340 and 6,350,579, each incorporated by reference herein in its entirety). Labels may be attached to a probe by various means including covalent linkages, chelation, and ionic interactions, but preferably the label is covalently attached. For example, in some embodiments, a detection probe has an attached chemiluminescent label such as, e.g., an acridinium ester (AE) compound (see, e.g., U.S. Pat. Nos. 5,185,439; 5,639,604; 5,585,481; and 5,656,744; each incorporated by reference herein), which in typical variations is attached to the probe by a non-nucleotide linker (see, e.g., U.S. Pat. Nos. 5,585,481; 5,656,744; and 5,639,604, particularly at column 10, line 6 to column 11, line 3, and Example 8; each incorporated by reference herein in its entirety). In other embodiments, a detection probe comprises both a fluorescent label and a quencher, a combination that is particularly useful in fluorescence resonance energy transfer (FRET) assays. Specific variations of such detection probes include, e.g., a TaqMan detection probe (Roche Molecular Diagnostics) and a “molecular beacon” (see, e.g., Tyagi et al., Nature Biotechnol. 16:49-53, 1998; U.S. Pat. Nos. 5,118,801 and 5,312,728; each incorporated by reference herein in its entirety).

A detection probe may further include a non-target-hybridizing sequence. Specific embodiments of such detection probes include, for example, probes that form conformations held by intramolecular hybridization, such as conformations generally referred to as hairpins. Particularly suitable hairpin probes include a “molecular torch” (see, e.g., U.S. Pat. Nos. 6,849,412; 6,835,542; 6,534,274; and 6,361,945, each incorporated by reference herein in its entirety) and a “molecular beacon” (see, e.g., Tyagi et al., supra; U.S. Pat. Nos. 5,118,801 and 5,312,728, supra). Methods for using such hairpin probes are well known in the art.

In particular embodiments, each of one or more detection probes for detecting one or more stx amplification products includes a fluorescent label (“fluorescent dye compound”). Suitable fluorophores are well-known in the art and include, for example, CalO 560, CalRed 610, and FAM. In some variations of amplification oligomers for determining the presence or absence of each of the stx variant target nucleic acids in a sample, detection probes specific for each stx variant target region is labeled with a different fluorophore. In other variations of amplification oligomers for determining the presence or absence of each of the stx variant target nucleic acids in a sample, detection probes specific for some of the stx variant target regions are labeled with the same fluorophore.

In particular embodiments, each of one or more forward amplification oligomers (e.g., forward primers) or each of one or more reverse amplification oligomers (e.g., reverse primers) includes a fluorescent label. This allows for each stx target nucleic acid amplification product to comprise a fluorescent label that is incorporated from a stx-specific amplification oligomer. In some variations of amplification oligomers for determining the presence or absence of each of the stx variant target nucleic acids in a sample, a fluorescent amplification oligomer specific for each stx variant target region is labeled with a different fluorophore. In other variations of amplification oligomers for determining the presence of absence of each of the stx variant target nucleic acids in a sample, fluorescent amplification oligomer specific for each stx variant target region is labeled with the same fluorophore. In some embodiments, the amplification oligomers comprising a fluorescent label comprise a sequence substantially corresponding to, or consisting of nucleotide sequences of (i) SEQ ID NOs: 29, 5, 23, and 26; ii) SEQ ID NOs: 14 and 15; (iii) SEQ ID NOs: 21 and 19; (iv) SEQ ID NOs: 31 and 27; (v) SEQ ID NO: 36; or (vi) SEQ ID NOs: 3, 4, 1, and 2. In some embodiments, the amplification oligomers comprising a fluorescent label comprise a sequence substantially corresponding to, or consisting of nucleotide sequences of (i) SEQ ID NOs: 33 and 40; (ii) SEQ ID NOs: 30 and 20; or (iii) SEQ ID NOs: 18, 30, and 20. In some embodiments, two or more of the stx-specific amplification oligomers comprise a fluorescent label so that each amplification product corresponding to a stx target region comprises a fluorescent label incorporated from a stx-specific amplification oligomer. This can be achieved, for example, by providing a label on each amplification oligomer oriented in a given direction (e.g., each forward amplification oligomer, or each reverse amplification oligomer). Unless indicated otherwise, amplification oligomers with a sequence corresponding to the sense strand of an stx target gene are considered forward amplification oligomers, and amplification oligomers with a sequence corresponding to the reverse complement of the sense strand of an stx target gene are considered reverse amplification oligomers.

In some embodiments, a detection probe or an amplification oligomer is labeled with CalO 560, CalRed 610, FAM, or ATTO 490LS. In some such embodiments comprising fluorophore-labeled detection probes or amplification oligomers, the detection probe(s) or amplification oligomer(s) further includes a quencher. Suitable quenchers are well-known in the art and include, for example, BHQ, TAMRA, and DABCLY.

### IV. Methods for Amplification and Detection of Nucleic Acids

A method for determining the presence or absence of a stx target nucleic acid generally includes the following steps: (1) contacting a sample suspected of containing at least one stx variant target nucleic acid with at least two amplification oligomers as described above for amplification of at least one stx variant target nucleic acid region; (2) performing an in vitro nucleic acid amplification reaction, where any stx variant target nucleic acid, if present in the sample, is used as a template for generating one or more amplification products corresponding to one or more of any stx variant target nucleic acids present in the sample; and (3) either (i) determining the sequences of the one or more amplification products or (ii) detecting the presence or absence of the one or more amplification products using one or more detection probes as described above for one or more stx target nucleic acid regions. In some embodiments, amplification oligomers for at least two stx variant targets are used in the method. In particular variations where amplification oligomers for at least two, three, or more stx variants are used, the method is performed as a multiplex assay. In some preferred embodiments, the detection step utilizes one or more detection probes for one or more stx variant target nucleic acids.

In certain embodiments, the method further includes purifying the stx target nucleic acid from other components in the sample before the contacting step (1). Such purification may include methods of separating and/or concentrating organisms contained in a sample from other sample components. In particular embodiments, purifying the stx target nucleic acid includes capturing the stx target nucleic acid to specifically or non-specifically separate the stx target nucleic acid from other sample components. Non-specific target capture methods may involve selective precipitation of nucleic acids from a substantially aqueous mixture, adherence of nucleic acids to a support that is washed to remove other sample components, or other means of physically separating nucleic acids from a mixture that contains stx target nucleic acid and other sample components. In some embodiments, stx target nucleic acid is selectively separated from other sample components by hybridizing the stx target nucleic acid to one or more capture probe oligomers. Compositions and methods for isolating nucleic acids are known and described in the literature (see, e.g., U.S. Pat. Nos. 5,234,809; 5,599,667; 6,110,678; 8,034,554; & 9,051,601; and international application WO 2018/209068, each incorporated by reference herein in its entirety).

Amplifying stx target nucleic acid sequences utilizes an in vitro amplification reaction using at least two amplification oligomers that flank a target region to be amplified. In some embodiments, the target region to be amplified substantially corresponds to a region ranging anywhere from about 50 to about 300 bases in length in any one of SEQ ID NOS: 46-58. Particularly suitable amplification oligomer combinations for amplification of these target regions are described herein. Suitable amplification methods include, for example, polymerase chain reaction (PCR), real-time polymerase chain reaction (RT-PCR), replicase-mediated amplification, ligase chain reaction (LCR), strand-displacement amplification (SDA), and transcription-associated amplification (e.g., TMA or NASBA). Such amplification methods are well-known in the art (see, e.g., the paragraphs defining “amplification” and “real-time amplification,” supra) and are readily used in accordance with the methods of the present disclosure.

Detection of the amplified products may be accomplished by a variety of methods. In some embodiments, labeled (e.g., fluorescently labeled) amplification oligomers are used that incorporate a label into amplified products. Such labeled products may then be detected through hybridization to probes (which need not be labeled) immobilized on a solid support and detection of signal associated with the solid support (e.g., detection of fluorescence, such as fluorometry or visualization, or via a light sensor such as a photomultiplier tube). In another embodiment, the nucleic acids may be associated with a surface that results in a physical change, such as a detectable electrical change. Amplified nucleic acids may be detected by concentrating them in or on a matrix and detecting the nucleic acids or dyes associated with them (e.g., an intercalating agent such as ethidium bromide or cyber green), or detecting an increase in dye associated with nucleic acid in solution phase. Other methods of detection may use nucleic acid detection probes that are configured to specifically hybridize to a sequence in the amplified product and detecting the presence of the probe:product complex, or by using a complex of probes that may amplify the detectable signal associated with the amplified products (e.g., U.S. Pat. Nos. 5,424,413; 5,451,503; 5,849,481; RE46,165; and 10,654,039; each incorporated by reference herein in its entirety). Directly or indirectly labeled probes that specifically associate with the amplified product provide a detectable signal that indicates the presence of the target nucleic acid in the sample.

Detection probes that hybridize to the complementary amplified sequences may be DNA or RNA oligomers, or oligomers that contain a combination of DNA and RNA nucleotides, or oligomers synthesized with a modified backbone, e.g., an oligomer that includes one or more 2′-methoxy substituted ribonucleotides. Probes used for detection of the amplified stx sequences may be unlabeled and detected indirectly (e.g., by binding of another binding partner to a moiety on the probe) or may be labeled with a variety of detectable labels. Particular embodiments of detection probes suitable for use in accordance with methods of the present disclosure are further described elsewhere herein. In some preferred embodiments of the method for detecting stx sequences, such as in certain embodiments using real-time polymerase chain reaction (RT-PCR), the detection probe is an oligomer comprising both a fluorescent label and a quencher (e.g., a TaqMan detection probe).

In some embodiments, a method for detecting the presence or absence of one or more stx target nucleic acid sequences as described herein further includes the detection of one or more other target microorganisms such as, for example, one or more other gastrointestinal pathogens. Detection of the presence or absence of one or more stx target nucleic acid sequences determines the presence or absence of one or more pathogens in a sample. In particular embodiment, a method as described herein further includes detecting the presence or absence of a Shiga-toxin-producing E. coli (STEC) such as, e.g., by amplification of a target region within a stx1, stx2a, stx2b, stx2c, stx2d, stx2e, stx2f, stx2g, stx2h, stx2i, stx2j, stx2k, stx21, and/or stx2m gene and detection of a corresponding amplification product. In a particular embodiment, a method as described herein further includes detecting the presence or absence of a Shiga-toxin-producing Shigella such as, e.g., by amplification of a target region within a stx gene. Detection of any one of STEC stx1, STEC stx2a, STEC stx2b, STEC stx2c, STEC stx2d, STEC stx2e, STEC stx2f, STEC stx2g, STEC stx2h, STEC stx2i, STEC stx2j, STEC stx2k, STEC stx21, STEC stx2m, Shigella stx, and/or any stx gene may be performed as a separate amplification/detection reaction from a multiplex reaction for detection of two or more stx as described herein. For example, a method may include a first multiplex reaction for determining the presence or absence of one or more stx as described herein and a second multiplex reaction for determining the presence or absence of other one or more stx.

### V. Reaction Mixtures

Also provided herein are reaction mixtures for amplification and/or detection of a stx target nucleic acid. In some embodiments, a reaction mixture comprises one or more of the following: an oligomer combination as described herein for amplification of a stx target nucleic acid; and a detection probe oligomer as described herein for determining the presence or absence of a stx amplification product. The reaction mixture may further include a number of optional components such as, for example, arrays of capture probe nucleic acids. For an amplification reaction mixture, the reaction mixture will typically include other reagents suitable for performing in vitro amplification such as, e.g., buffers, salt solutions, appropriate nucleotide triphosphates (e.g., dATP, dCTP, dGTP, dTTP, ATP, CTP, GTP and UTP), and/or enzyme(s) (e.g., DNA polymerase, reverse transcriptase, RNA polymerase), and may include test sample components, in which a stx target nucleic acid may or may not be present. In addition, for a reaction mixture that includes a detection probe together with an amplification oligomer combination, selection of amplification oligomers and detection probe oligomers for a reaction mixture are linked by a common target region (e.g., the reaction mixture will include a probe that binds to a sequence amplifiable by an amplification oligomer combination of the reaction mixture).

### VI. Kits

Also provided herein are kits for practicing the methods as described herein. In some embodiments, a kit comprises one or more of the following: an oligomer combination or a set of oligomers as described herein for amplification of a stx target nucleic acid; and a detection probe oligomer as described herein for determining the presence or absence of a stx amplification product. The kits may further include a number of optional components such as, for example, arrays of capture probe nucleic acids. Other reagents that may be present in the kits include reagents suitable for performing in vitro amplification such as, e.g., buffers, salt solutions, appropriate nucleotide triphosphates (e.g., dATP, dCTP, dGTP, dTTP, ATP, CTP, GTP and UTP), and/or enzyme(s) (e.g., DNA polymerase, reverse transcriptase, RNA polymerase). Oligomers as described herein may be packaged in a variety of different embodiments, and those skilled in the art will appreciate that the instant disclosure embraces many different kit configurations. For example, a kit may include amplification oligomers for only one stx variant target region, or it may include amplification oligomers for two or more stx variant target regions. In addition, for a kit that includes a detection probe together with an amplification oligomer combination, selection of amplification oligomers and detection probe oligomers for a kit are linked by a common target region (e.g., the kit will include a probe that binds to a region amplifiable by an amplification oligomer combination of the kit). In certain embodiments, the kit further includes a set of instructions for practicing methods in accordance with the present disclosure, where the instructions may be associated with a package insert and/or the packaging of the kit or the components thereof.

In some embodiments, the kit comprises a microarray cartridge for the detection and amplification of stx in a sample. In some embodiments, the microarray cartridge contains reagents for sample preparation, PCR amplification of target nucleic acids, and hybridization of amplified nucleic acids. The microarray cartridge may contain reagents for the preparation and analysis of one or more samples.

In some embodiments, the sample is a stool sample obtained from a subject suspected of having bacterial and/or stx. Before the sample is applied to the microarray cartridge, the sample may be subjected to mechanical breakage, e.g., by bead beating or sonication, such that target nucleic acids may be obtained from bacteria in the sample. In some embodiments, the microarray cartridge is used to perform extraction and purification of target nucleic acids.

The microarray cartridge may comprise an internal control (IC), which are control nucleic acids subjected to the same preparation steps as the target nucleic acids. Target nucleic acids and control nucleic acids are transferred to two independent PCR chambers where both the target nucleic acids and the control nucleic acids are amplified. The IC or PC is detected with amplicon-specific fluorophore labeled probes.

The microarray cartridge may comprise any combination of amplification oligomers (e.g., forward and reverse primers) disclosed herein. In some embodiments, the forward primer and/or reverse primer is labeled with a detection label.

Detection oligomers (e.g., hybridization probes) and gridding controls may be printed on the microarray cartridge. In many embodiments, the microarray cartridge comprises any combination of the detection oligomers disclosed herein. The presence of stx DNA is determined by the detection of a set of detection probes which hybridize to specific stx variants, as disclosed herein. In some embodiments, an integrated software algorithm is used to analyze and/or display real-time PCR signal and/or microarray image.

### VII. Samples

Also provided herein are examples of samples that may be used with the methods, oligomers, reaction mixtures, and/or kits of the present disclosure. In some embodiments, a sample, such as stool, is obtained from a subject suspected of being infected with a pathogen that produces stx. In some embodiments, the sample is food or water suspected of containing a pathogen that produces stx.

The invention is further illustrated by the following non-limiting examples.

### Example 1

Primer Sensitivity for E. coli stx2 Variants

### I. Objective

This example demonstrates sensitivity of PCR primers for amplifying E. coli stx2 variants: stx2a, stx2b, stx2c, stx2d, stx2e, stx2f stx2g, stx2h, stx2i, and stx2k.

### II. Materials and Methods

Tagman™ Real-time PCR reactions were performed. The reactions used forward and reverse primers for amplifying a stx2 variant DNA template, and fluorescent nucleic acid probes that are complementary to an internal segment of a stx2 variant DNA. The probes comprised FAM-labeled probes and one or more quenchers (e.g., with the double quenchers ZEN quencher and 5′ Iowa Black® FQ). DNA templates were stx2 DNA sequences from enterohemorrhagic E. coli (EHEC). Signal detection was achieved through monitoring an increase in fluorescence as the amplification reactions proceed.

The following cycling parameters were used: 95° C. for 10 min (optics off), 45 cycles of 95° C. for 15 sec (optics off), 60° C. for 60 sec (optics on). Table 1 below provides reagent volumes and concentrations used in the reactions. Table 2 below provides the different combinations of primers and probes in the assay mixes used in the reactions. The Control/Reference Assay Mix used was an assay mix with known forward and reverse primers typically used to amplify stx2. The reactions were setup in a 96-well plate as shown in Table 3 below. As indicated in Table 3, (1) DNA templates for stx2a, stx2b, stx2c, stx2d, stx2e, stx2f stx2g, stx2h, stx2i, and stx2k were used in plate columns 1, 2, 3, 4, 5, 6, 7, 8, 9, and 10, respectively; and (2) assay mixes 1, 2, 3, 4, 5, and 6 were used in plate rows A, B, C, D, E, F, G, and H, respectively. In Table 3, the label for each sample indicates the stx2 variant DNA template and the amount of DNA template used. For example, “stx2a SID 46 10k c.” in well A1 indicates that the stx2a template with the nucleotide sequence of SEQ ID NO: 46 was used, and there were 10,000 copies of that template in the reaction. Nucleotide sequences of DNA templates are provided in Table 6 below.

TABLE 1
Reagents Used in the Assay for Detecting E. coli stx2 Variants.

| | Stock | Reaction | | Volume |
|---|---|---|---|---|
| | Concen- | Concen- | Volume | for 90 |
| Reagent | tration | tration | (μL) | Reactions (μL) |
| 2x Real-Time | 2x | 1x | 10 | 900.00 |
| PCR Master Mix | | | | |
| 10x Assay Mix | 4 μM each | 0.4 μM | 2.00 | 180.00 |
| for each of | oligomer | | | |
| Assays 1-6 | | | | |
| MilliQ water | | | 6.00 | 540.00 |
| Template DNA | | Mix total | 18 | 1620.00 |
| | | | 2 | |
| | | Reaction total | 20.0 | |

TABLE 2
Combinations of Primers and Probes for Assay Mixes 1-6.

| | |
|---|---|
| Description | SEQ ID NO |

Assay Mix 1

| | |
|---|---|
| Forward primer | 29 |
| Forward primer | 5 |
| Forward primer | 23 |
| Forward primer | 26 |
| Reverse primer | 33 |
| Reverse primer | 40 |
| Probe | 28 |

Assay Mix 2

| | |
|---|---|
| Forward primer | 14 |
| Forward primer | 15 |
| Reverse primer | 18 |
| Reverse primer | 30 |
| Reverse primer | 20 |
| Probe | 28 |

Assay Mix 3

| | |
|---|---|
| Forward primer | 21 |
| Forward primer | 19 |
| Reverse primer | 18 |
| Reverse primer | 30 |
| Reverse primer | 20 |
| Probe | 28 |

Assay Mix 4

| | |
|---|---|
| Forward primer | 31 |
| Forward primer | 27 |
| Reverse primer | 18 |
| Reverse primer | 30 |
| Reverse primer | 20 |
| Probe | 28 |

Assay Mix 5

| | |
|---|---|
| Forward primer | 36 |
| Forward primer | 27 |
| Reverse primer | 18 |
| Reverse primer | 30 |
| Reverse primer | 20 |
| Probe | 28 |

Assay Mix 6

| | |
|---|---|
| Forward primer | 03 |
| Forward primer | 04 |
| Forward primer | 01 |
| Forward primer | 02 |
| Reverse primer | 18 |
| Reverse primer | 30 |
| Reverse primer | 20 |
| Probe | 28 |

Control/Reference Assay Mix

| | |
|---|---|
| Forward primer | 37 |
| Forward primer | 06 |
| Reverse primer | 24 |
| Reverse primer | 41 |
| Probe | 32 |
| Probe | 34 |
| Probe | 35 |

TABLE 3
Layout of Samples in a 96-well Plate for PCR Reactions.

| | 1 | 2 | 3 | 4 | 5 | 6 | 7 | 8 | 9 | 10 |
|---|---|---|---|---|---|---|---|---|---|---|
| A | stx2a | stx2b | stx2c | stx2d | stx2e | stx2f | stx2g | stx2h | stx2i | stx2k |
| | SID 46 | SID 47 | SID 48 | SID 49 | SID 50 | SID 51 | SID 52 | SID 53 | SID 54 | SID 56 |
| | 10k c. | 10k c. | 10k c. | 10k c. | 10k c. | 10k c. | 10k c. | 10k c. | 10k c. | 10k c. |
| B | stx2a | stx2b | stx2c | stx2d | stx2e | stx2f | stx2g | stx2h | stx2i | stx2k |
| | SID 46 | SID47 | SID 48 | SID 49 | SID 50 | SID 51 | SID 52 | SID 53 | SID 54 | SID 56 |
| | 1000 c. | 1000 c. | 1000 c. | 1000 c. | 1000 c. | 1000 c. | 1000 c. | 1000 c. | 1000 c. | 1000 c. |
| C | stx2a | stx2b | stx2c | stx2d | stx2e | stx2f | stx2g | stx2h | stx2i | stx2k |
| | SID 46 | SID471 | SID 48 | SID 49 | SID 50 | SID 51 | SID 52 | SID 53 | SID 54 | SID 56 |
| | 100 c. | 00 c. | 100 c. | 100 c. | 100 c. | 100 c. | 100 c. | 100 c. | 100 c. | 100 c. |
| D | stx2a | stx2b | stx2c | stx2d | stx2e | stx2f | stx2g | stx2h | stx2i | stx2k |
| | SID 46 | SID47 | SID 48 | SID 49 | SID 50 | SID 51 | SID 52 | SID 53 | SID 54 | SID 56 |
| | 10 c. | 10 c. | 10 c. | 10 c. | 10 c. | 10 c. | 10 c. | 10 c. | 10 c. | 10 c. |
| E | stx2a | stx2b | stx2c | stx2d | stx2e | stx2f | stx2g | stx2h | stx2i | stx2k |
| | SID 46 | SID47 | SID 48 | SID 49 | SID 50 | SID 51 | SID 52 | SID 53 | SID 54 | SID 56 |
| | 10k c. | 10k c. | 10k c. | 10k c. | 10k c. | 10k c. | 10k c. | 10k c. | 10k c. | 10k c. |
| F | stx2a | stx2b | stx2c | stx2d | stx2e | stx2f | stx2g | stx2h | stx2i | stx2k |
| | SID 46 | SID471 | SID 48 | SID 49 | SID 50 | SID 51 | SID 52 | SID 53 | SID 54 | SID 56 |
| | 1000 c. | 000 c. | 1000 c. | 1000 c. | 1000 c. | 1000 c. | 1000 c. | 1000 c. | 1000 c. | 1000 c. |
| G | stx2a | stx2b | stx2c | stx2d | stx2e | stx2f | stx2g | stx2h | stx2i | stx2k |
| | SID 46 | SID47 | SID 48 | SID 49 | SID 50 | SID 51 | SID 52 | SID 53 | SID 54 | SID 56 |
| | 100 c. | 100 c. | 100 c. | 100 c. | 100 c. | 100 c. | 100 c. | 100 c. | 100 c. | 100 c. |
| H | stx2a | stx2b | stx2c | stx2d | stx2e | stx2f | stx2g | stx2h | stx2i | stx2k |
| | SID 46 | SID47 | SID 48 | SID 49 | SID 50 | SID 51 | SID 52 | SID 53 | SID 54 | SID 56 |
| | 10 c | 10 c. | 10 c. | 10 c. | 10 c. | 10 c. | 10 c. | 10 c. | 10 c. | 10 c. |

### III. Results

As shown by fluorescence units, Cq, in Table 4 below, variants stx2a through stx2i and stx2k were successfully amplified using Assay Mixes 1-6. In contrast, the Control/Reference Assay Mix only amplified variants stx2a through stx2g and not stx2h, stx2i, and stx2k, as shown by insufficient Cq for detection. In Table 4, the label for each sample indicates the stx2 variant DNA template and the amount of DNA template used. For example, “stx2a SID 46 10k c.” indicates that the stx2a template with the nucleotide sequence of SEQ ID NO: 46 was used, and there were 10,000 copies of that template in the reaction. Nucleotide sequences of DNA templates are provided in Table 6 below.

TABLE 4
Detection of E. coli stx2 Variants.

| | | | | | | | |
|---|---|---|---|---|---|---|---|
| | Control | Assay 1 | Assay 2 | Assay 3 | Assay 4 | Assay 5 | Assay 6 |
| Sample | Cq | Cq | Cq | Cq | Cq | Cq | Cq |

stx2a

| | | | | | | | |
|---|---|---|---|---|---|---|---|
| stx2a SID 46 10k | 25.82 | 25.9 | 25.83 | 25.57 | 25.67 | 25.37 | 25.19 |
| c. | | | | | | | |
| stx2a SID 46 10k | 25.72 | 25.89 | 25.98 | 25.7 | 25.57 | 25.66 | 25.39 |
| c. | | | | | | | |
| stx2a SID 46 | 29.69 | 29.97 | 29.97 | 29.26 | 29.78 | 30.01 | 29.77 |
| 1000 c. | | | | | | | |
| stx2a SID 46 | 29.38 | 29.79 | 30.64 | 29.11 | 29.82 | 28.83 | 28.81 |
| 1000 c. | | | | | | | |
| stx2a SID 46 100 | 33.07 | 32.92 | 33.59 | 33.47 | 32.77 | 33.62 | 33.45 |
| c. | | | | | | | |
| stx2a SID 46 100 | 32.57 | 32.52 | 35.33 | 33.1 | 33.2 | 32.46 | 32.47 |
| c. | | | | | | | |
| stx2a SID 46 10 c. | 35.87 | 35.78 | 36.11 | 35.67 | 36.68 | 35.46 | 39.02 |
| stx2a SID 46 10 c. | 36.68 | 34.41 | 38.11 | 36.7 | 36.12 | 34.21 | 35.31 |

stx2b

| | | | | | | | |
|---|---|---|---|---|---|---|---|
| stx2b SID 47 10k | 25.07 | 25.09 | 25.2 | 24.86 | 24.81 | 24.6 | 24.26 |
| c. | | | | | | | |
| stx2b SID 47 10k | 24.93 | 25.28 | 25.03 | 24.77 | 24.79 | 24.83 | 24.45 |
| c. | | | | | | | |
| stx2b SID 47 | 28.55 | 28.84 | 30.25 | 28.65 | 29.17 | 30.12 | 28.51 |
| 1000 c. | | | | | | | |
| stx2b SID 47 | 28.63 | 28.72 | 30.52 | 28.84 | 28.79 | 29.15 | 27.96 |
| 1000 c. | | | | | | | |
| stx2b SID 47 100 | 32.31 | 32.13 | 33.84 | 32.11 | 32.69 | 34.05 | 32.41 |
| c. | | | | | | | |
| stx2b SID 47 100 | 31.69 | 32.34 | 34.02 | 32.37 | 31.62 | 33.15 | 32.07 |
| c. | | | | | | | |
| stx2b SID 45 10 | 36.27 | 35.96 | 36.23 | 35.34 | 34.08 | 35.86 | 35.44 |
| c. | | | | | | | |
| stx2b SID 47 10 | 35.21 | 34.73 | 37.35 | 36.42 | 33.75 | 37.27 | 35.29 |
| c. | | | | | | | |

stx2c

| | | | | | | | |
|---|---|---|---|---|---|---|---|
| stx2c SID 48 10k | 25.43 | 25.45 | 25.76 | 25.52 | 25.38 | 25.35 | 24.79 |
| c. | | | | | | | |
| stx2c SID 4810k | 25.46 | 25.44 | 25.75 | 25.33 | 25.3 | 25.54 | 25.09 |
| c. | | | | | | | |
| stx2c SID 48 | 29.24 | 29.22 | 29.67 | 29.06 | 29.35 | 28.61 | 28.95 |
| 1000 c. | | | | | | | |
| stx2c SID 48 | 29.23 | 28.99 | 30.43 | 29.09 | 29.34 | 28.68 | 28.58 |
| 1000 c. | | | | | | | |
| stx2c SID 48 100 | 32.5 | 32.63 | 33.18 | 32.52 | 32.56 | 33.29 | 32.81 |
| c. | | | | | | | |
| stx2c SID 48 100 | 32.39 | 33.01 | 33.39 | 32.67 | 32.47 | 31.91 | 32.26 |
| c. | | | | | | | |
| stx2c SID 48 10 c. | 35.46 | 35.48 | 36.59 | 35.66 | 36.02 | 34.87 | 35.91 |
| stx2c SID 48 10 c. | 34.94 | 35.07 | 35.99 | 36.15 | 35.17 | 35.07 | 34.97 |

stx2d

| | | | | | | | |
|---|---|---|---|---|---|---|---|
| stx2d SID 49 10k | 23.54 | 23.63 | 23.87 | 23.33 | 23.33 | 23.34 | 23.09 |
| c. | | | | | | | |
| stx2d SID 49 10k | 23.34 | 23.49 | 23.66 | 23.36 | 23.28 | 23.34 | 23.02 |
| c. | | | | | | | |
| stx2d SID 49 | 27.19 | 27.39 | 27.96 | 27.41 | 27.55 | 28.18 | 27.83 |
| 1000 c. | | | | | | | |
| stx2d SID 47 | 27.27 | 27.39 | 28.5 | 28.35 | 27.14 | 26.88 | 26.66 |
| 1000 c. | | | | | | | |
| stx2d SID 49 100 | 30.6 | 31.17 | 31.12 | 31.48 | 30.63 | 32.37 | 31.11 |
| c. | | | | | | | |
| stx2d SID 49 100 | 30.43 | 30.98 | 31.86 | 31.56 | 30.06 | 30.1 | 30.38 |
| c. | | | | | | | |
| stx2d SID 49 10 | 33.54 | 34.04 | 34.65 | 33.67 | 34.12 | 34.22 | 33.97 |
| c. | | | | | | | |
| stx2d SID 49 10 | 33.1 | 33.04 | 34.37 | 33.69 | 33.09 | 34.12 | 32.81 |
| c. | | | | | | | |

stx2e

| | | | | | | | |
|---|---|---|---|---|---|---|---|
| stx2e SID 50 10k | 23.66 | 23.92 | 23.75 | 23.5 | 23.19 | 23.47 | 23.16 |
| c. | | | | | | | |
| stx2e SID 50 10k | 23.75 | 23.87 | 23.81 | 23.49 | 23.52 | 23.68 | 23.28 |
| c. | | | | | | | |
| stx2e SID 50 | 27.13 | 27.44 | 27.41 | 27.04 | 27.07 | 26.81 | 27.33 |
| 1000 c. | | | | | | | |
| stx2e SID 50 | 27.23 | 27.06 | 28.24 | 28.15 | 27.21 | 25.6 | 26.12 |
| 1000 c. | | | | | | | |
| stx2e SID 50 100 | 30.56 | 31.71 | 30.51 | 30.6 | 30.32 | 30.49 | 30.89 |
| c. | | | | | | | |
| stx2e SID 50 100 | 30.58 | 31.82 | 31.25 | 31.66 | 30.52 | 30.36 | 29.49 |
| c. | | | | | | | |
| stx2e SID 50 10 c. | 33.85 | 35.06 | 34.07 | 32.72 | 32.94 | 33.39 | 33.57 |
| stx2e SID 50 10 c. | 33.36 | 33.84 | 34.24 | 34.55 | 33.68 | 33.6 | 32.05 |

stx2f

| | | | | | | | |
|---|---|---|---|---|---|---|---|
| stx2f SID 5110k | 23.4 | 23.6 | 24.08 | 23.74 | 23.58 | 36.29 | 23.01 |
| c. | | | | | | | |
| stx2f SID 51 10k | 23.49 | 23.63 | 24.3 | 23.84 | 23.6 | 36.13 | 23.46 |
| c. | | | | | | | |
| stx2f SID 51 1000 | 26.86 | 27.6 | 28.48 | 27.08 | 27.79 | 41 | 26.85 |
| c. | | | | | | | |
| stx2f SID 51 1000 | 27.28 | 29.28 | 27.83 | 27.66 | 27.42 | 40.06 | 27.17 |
| c. | | | | | | | |
| stx2f SID 51 100 | 30.2 | 31.63 | 31.86 | 32.14 | 31.09 | 0 | 29.83 |
| c. | | | | | | | |
| stx2f SID 51 100 | 30.36 | 32.59 | 31.39 | 31.54 | 30.79 | 0 | 32.38 |
| c. | | | | | | | |
| stx2f SID 51 10 c. | 33.36 | 34.37 | 33.88 | 35.2 | 34.46 | 0 | 33.34 |
| stx2f SID 49 10 c. | 33.19 | 35.16 | 33.42 | 34.41 | 33.45 | 0 | 34.26 |

stx2g

| | | | | | | | |
|---|---|---|---|---|---|---|---|
| stx2g SID 52 10k | 26.7 | 23.74 | 23.76 | 23.48 | 23.4 | 23.75 | 23.1 |
| c. | | | | | | | |
| stx2g SID 52 10k | 26.82 | 23.92 | 23.8 | 23.52 | 23.45 | 23.44 | 23.19 |
| c. | | | | | | | |
| stx2g SID 52 | 30.67 | 27.77 | 27.73 | 26.88 | 27.2 | 28.82 | 26.34 |
| 1000 c. | | | | | | | |
| stx2g SID 52 | 30.85 | 27.66 | 27.69 | 27.16 | 27.12 | 26.33 | 28.16 |
| 1000 c. | | | | | | | |
| stx2g SID 52 100 | 34.11 | 31.58 | 30.91 | 30.45 | 30.7 | 32.7 | 29.82 |
| c. | | | | | | | |
| stx2g SID 52 100 | 33.94 | 31.05 | 30.91 | 30.45 | 30.42 | 30.16 | 32.27 |
| c. | | | | | | | |
| stx2g SID 52 10 | 37.61 | 34.07 | 33.64 | 34.45 | 33.59 | 35.38 | 32.31 |
| c. | | | | | | | |
| stx2g SID 50 10 | 37.45 | 33.68 | 34.2 | 33.61 | 33.27 | 32.64 | 35.66 |
| c. | | | | | | | |

stx2h

| | | | | | | | |
|---|---|---|---|---|---|---|---|
| stx2h SID 53 10k | Below | 24.6 | 24.48 | 24.15 | 24.24 | 24.2 | 25.13 |
| c. | the limit | | | | | | |
| | of | | | | | | |
| | detection | | | | | | |
| stx2h SID 53 10k | Below | 24.81 | 24.45 | 24.28 | 24.23 | 24.27 | 25.51 |
| c. | the limit | | | | | | |
| | of | | | | | | |
| | detection | | | | | | |
| stx2h SID 53 | Below | 29.17 | 29.49 | 28.46 | 28.15 | 27.46 | 28.38 |
| 1000 c. | the limit | | | | | | |
| | of | | | | | | |
| | detection | | | | | | |
| stx2h SID 53 | Below | 28.62 | 28.89 | 28.01 | 28 | 26.83 | 30.07 |
| 1000 c. | the limit | | | | | | |
| | of | | | | | | |
| | detection | | | | | | |
| stx2h SID 53 100 | Below | 32.74 | 34.69 | 32.6 | 31.47 | 31.62 | 31.68 |
| c. | the limit | | | | | | |
| | of | | | | | | |
| | detection | | | | | | |
| stx2h SID 53 100 | Below | 32.1 | 32.01 | 31.54 | 31.26 | 30.32 | 34.46 |
| c. | the limit | | | | | | |
| | of | | | | | | |
| | detection | | | | | | |
| stx2h SID 53 10 | Below | 35.27 | 36.63 | 36.21 | 34.35 | 33.71 | 34.18 |
| c. | the limit | | | | | | |
| | of | | | | | | |
| | detection | | | | | | |
| stx2h SID 53 10 | Below | 34.19 | 35.02 | 34.56 | 34.12 | 33.03 | 37.82 |
| c. | the limit | | | | | | |
| | of | | | | | | |
| | detection | | | | | | |

stx2i

| | | | | | | | |
|---|---|---|---|---|---|---|---|
| stx2i SID 54 10k | Below | 24.05 | 24.33 | 23.95 | 23.9 | 23.9 | 23.26 |
| c. | the limit | | | | | | |
| | of | | | | | | |
| | detection | | | | | | |
| stx2i SID 54 10k | Below | 23.72 | 24.27 | 23.96 | 23.88 | 23.79 | 23.59 |
| c. | the limit | | | | | | |
| | of | | | | | | |
| | detection | | | | | | |
| stx2i SID 54 1000 | Below | 27.54 | 29.48 | 28.28 | 28.13 | 27.58 | 26.46 |
| c. | the limit | | | | | | |
| | of | | | | | | |
| | detection | | | | | | |
| stx2i SID 54 | Below | 27.99 | 28.36 | 28.23 | 28.08 | 27.22 | 26.55 |
| 1000 c. | the limit | | | | | | |
| | of | | | | | | |
| | detection | | | | | | |
| stx2i SID 54 100 | Below | 31.72 | 34.1 | 34.33 | 31.15 | 31.66 | 29.76 |
| c. | the limit | | | | | | |
| | of | | | | | | |
| | detection | | | | | | |
| stx2i SID 54 100 | Below | 32.9 | 32.15 | 31.31 | 31.12 | 30.57 | 31.05 |
| c. | the limit | | | | | | |
| | of | | | | | | |
| | detection | | | | | | |
| stx2i SID 5410 c. | Below | 35.04 | 37.25 | 36.44 | 34.27 | 34.78 | 32.64 |
| | the limit | | | | | | |
| | of | | | | | | |
| | detection | | | | | | |
| stx2i SID 54 10 c. | Below | 35.94 | 35.27 | 36.1 | 34.34 | 33.38 | 33.58 |
| | the limit | | | | | | |
| | of | | | | | | |
| | detection | | | | | | |

stx2k

| | | | | | | | |
|---|---|---|---|---|---|---|---|
| stx2k SID 56 10k | Below | 25.17 | 25.75 | 25.26 | 25.26 | 25.2 | 25 |
| c. | the limit | | | | | | |
| | of | | | | | | |
| | detection | | | | | | |
| stx2k SID 56 10k | Below | 25.31 | 25.73 | 25.48 | 25.28 | 25.2 | 25.04 |
| c. | the limit | | | | | | |
| | of | | | | | | |
| | detection | | | | | | |
| stx2k SID 56 | Below | 29.28 | 29.61 | 30.31 | 29.46 | 29.29 | 28.2 |
| 1000 c. | the limit | | | | | | |
| | of | | | | | | |
| | detection | | | | | | |
| stx2k SID 56 | Below | 29.06 | 29.33 | 29.71 | 29.62 | 0 | 29.2 |
| 1000 c. | the limit | | | | | | |
| | of | | | | | | |
| | detection | | | | | | |
| stx2k SID 56 100 | Below | 33.84 | 33.96 | 33.84 | 32.64 | 38.33 | 31.71 |
| c. | the limit | | | | | | |
| | of | | | | | | |
| | detection | | | | | | |
| stx2k SID 56 100 | Below | 34.06 | 32.62 | 33.56 | 32.68 | 0 | 32.46 |
| c. | the limit | | | | | | |
| | of | | | | | | |
| | detection | | | | | | |
| stx2k SID 56 10 | Below | 36.44 | 38.94 | 36.71 | 35.28 | 0 | 35.35 |
| c. | the limit | | | | | | |
| | of | | | | | | |
| | detection | | | | | | |
| stx2k SID 56 10 | Below | 37.49 | 36.73 | 37.49 | 36.19 | 0 | 35.73 |
| c. | the limit | | | | | | |
| | of | | | | | | |
| | detection | | | | | | |

### Example 2

Microarray Probe Sensitivity for E. coli Stx2 Variants

### I. Objective

This example demonstrates sensitivity of fluorescent probes for detecting E. coli stx2 variants: stx2a, stx2b, stx2c, stx2d, stx2e, stx2f stx2g, stx2h, stx2i, stx2j, and stx2k in a microarray.

### II. Materials and Methods

The Novodiag® Bacterial GE+ test was used as a testing platform for detection of stx2. The Novodiag® Bacterial GE+ assay, performed with the Novodiag System, is a cartridge-based molecular test for qualitative detection of bacterial gastroenteritis. The cartridge includes a cyclo olefin polymer microarray to which probes are attached.

The specific probe sequences used were as indicated in Tables 5A-5D below. Probes that target stx2 variant(s) were immobilized on an array. DNA templates were stx2 DNA sequences from enterohemorrhagic E. coli (EHEC).

PCR primers used in the amplification of the stx2 were as shown above for Assay Mix 1 (Table 2) accompanied by the multiplex primer mix of Novodiag® Bacterial GE+. Reverse primers used for amplification of stx2 variant(s) were labeled with the ATTO 490LS fluorescent dye. Thus, amplicons were labeled with ATTO 490LS by incorporation of the reverse primers. Amplicons were denatured to produce single-stranded DNA molecules, which hybridized to immobilized microarray probes. Fluorescence signal from labeled DNA molecules that hybridized to the probes were detected.

The following end-point PCR cycling parameters were used: 109° C. for 20 s, 99° C. for 2 min, 50 cycles of 109° C. for 5 sec, 57° C. for 15 sec.

The following hybridization conditions were used in a salt/detergent buffer: hybridization of the amplicon on a microarray for 10 min at 52° C.

### III. Results

As shown in Tables 5A-5D below, variants stx2A through stx2k were successfully amplified and detected using different probes on a microarray contained in a cartridge. The microarray contained five replicate spots for each stx probe. Microarray signal was recorded in relative fluorescence units (RFU). Variants with no disclosed RFUs indicate that the amplification and detection of those variants were below the limit of detection of the method. In tables 5A-D, for each stx2 variant, the three columns represent replicate cartridges. The five rows represent the five replicate spots on the microarray of the given probe.

Hybridization data showed that there are microarray probes effective for detecting each of the tested stx2 types. Each of the tested stx2 types was detected by at least one of the probes. Effectiveness of a probe is determined based on the signal level measured from the spots and several other spot parameters which enable spot detection. Effective probes for a given target showed multiple positive replicate spots.

TABLE 5A
Detection of Stx2a, Stx2b, and Stx2c by Microarray.

| Probe | | | | | | | | | |
|---|---|---|---|---|---|---|---|---|---|
| SEQ | Relative | | | | | | | | |
| ID | Fluorescence Units, | | | | | | | | |
| NO | RFU, for stx2c | | | RFU for stx2b | | | RFU for stx2a | | |
| 10 | 677 | 354 | 304 | 2168 | 2914 | 2882 | 214 | 409 | 405 |
| 10 | 574 | 432 | 404 | 2172 | 3316 | 2832 | 256 | 296 | 768 |
| 10 | 661 | 1014 | 498 | 2710 | 4073 | 4059 | | 1108 | 580 |
| 10 | 433 | 454 | 486 | 1857 | 2583 | 2550 | | 384 | 279 |
| 10 | | 205 | | 1493 | 2663 | 1214 | 159 | 268 | 256 |
| 8 | 2728 | 2131 | 2434 | 1011 | 1671 | 1994 | 2185 | 2496 | 2614 |
| 8 | 3511 | 4244 | 3023 | | 3154 | 2612 | 2895 | 3067 | 3514 |
| 8 | 2588 | 1531 | 2096 | 1033 | 1402 | 1763 | 2907 | 3452 | 2219 |
| 8 | 2013 | 2168 | 2274 | 1815 | 1815 | 1296 | 1991 | 2491 | 2837 |
| 8 | 2919 | 2655 | 3118 | 1555 | 1994 | 2316 | 2324 | 2348 | 1722 |
| 12 | 5341 | 4682 | 4479 | 648 | 426 | | 4429 | 5336 | 5633 |
| 12 | 3922 | 2473 | 3378 | 423 | 507 | 638 | 2543 | 3892 | 3662 |
| 12 | 3901 | 3423 | 3324 | 348 | 401 | 400 | 3050 | 4063 | 3569 |
| 12 | 4192 | 3288 | 3415 | 334 | 385 | 504 | 3179 | 3917 | 3849 |
| 12 | 4629 | 2272 | 2532 | | 388 | | 2369 | 5025 | 5284 |
| 7 | 1721 | 1165 | 1484 | 1205 | 1569 | 1949 | 1299 | 1663 | 1664 |
| 7 | 2066 | 1714 | 1652 | 1523 | 1914 | 2243 | 1657 | 1933 | 1896 |
| 7 | 2751 | 3023 | 2425 | | 2840 | 3434 | 3081 | 2570 | 2875 |
| 7 | 2167 | 1996 | 1584 | 1374 | 2101 | 2519 | 1636 | 2010 | 2210 |
| 7 | 1954 | 1510 | 1609 | 1324 | 1716 | 1838 | 1426 | 1937 | 1707 |
| 13 | 3210 | 2592 | 2548 | 1811 | 3036 | 3671 | 2654 | 2864 | 2578 |
| 13 | 1715 | 1386 | 1541 | 1196 | 1829 | 2011 | 1571 | 1656 | 1642 |
| 13 | 2121 | 2307 | 2065 | 1905 | 2214 | 2817 | 2529 | 1983 | 2378 |
| 13 | 1677 | 1212 | 1420 | 1316 | 1726 | 1942 | 1090 | 1501 | 1590 |
| 13 | 1736 | 1244 | 1324 | 1281 | 1646 | 1915 | 1217 | 1639 | 1624 |
| 9 | | | | | | | | | |
| 9 | | | | | | | | | 843 |
| 9 | | | | | | | | | |
| 9 | | | | | 477 | | | | |
| 9 | | | | | | 150 | | | |
| 11 | | | | | | | | | 204 |
| 11 | | | | | | | | | |
| 11 | | | | | | | | | |
| 11 | | | | | | | | | |
| 11 | | | | | | | | | |

TABLE 5B
Detection of Stx2d, Stx2e, and Stx2f by Microarray.

| Probe | | | | | | | | | |
|---|---|---|---|---|---|---|---|---|---|
| SEQ | | | | | | | | | |
| ID NO | RFU for stx2f | | | RFU for stx2e | | | RFU for stx2d | | |
| 10 | | | | 2452 | 2902 | 913 | 322 | 443 | 156 |
| 10 | | | | 2675 | 3544 | 750 | 273 | 647 | 482 |
| 10 | | | | 4270 | 3746 | 1129 | 776 | 1538 | 1090 |
| 10 | | | | 2633 | 4240 | 436 | 276 | 491 | 262 |
| 10 | | | | 2107 | 2882 | 434 | 450 | 445 | 207 |
| 8 | | 1175 | | 1535 | 1736 | 535 | 2381 | 2787 | 2304 |
| 8 | | | | 1443 | 2193 | 878 | 2665 | 6014 | 4680 |
| 8 | | | | 1344 | 1434 | | 1971 | 2461 | 1869 |
| 8 | 161 | | | 2395 | 1801 | 190 | 2324 | 2185 | 2529 |
| 8 | 563 | | | 1805 | 2067 | 465 | 2733 | 2654 | 3788 |
| 12 | | | | 329 | 661 | 143 | 4537 | 5838 | 5206 |
| 12 | | | | 357 | 340 | 126 | 3520 | 4274 | 3197 |
| 12 | | | | 207 | 355 | 111 | 3380 | 3711 | 3846 |
| 12 | | | | 302 | 293 | 119 | 3688 | 6577 | 4561 |
| 12 | | | | 1802 | | | 2727 | 2423 | 2907 |
| 7 | | | | 1775 | 1698 | 586 | 1394 | 1774 | 1510 |
| 7 | | | | 1922 | 2241 | 462 | 2092 | | 2042 |
| 7 | | | | 2151 | 3160 | 511 | 4873 | 3051 | 3630 |
| 7 | | | | 3100 | 2199 | | 3825 | 2129 | 2861 |
| 7 | | | | 1900 | 1893 | 719 | 1698 | 1855 | 1424 |
| 13 | | | | 3139 | 3057 | 982 | 3076 | 3208 | 3233 |
| 13 | | | | 1972 | 1994 | 475 | 1756 | 1495 | 1908 |
| 13 | | | | 1930 | 2260 | 341 | 2062 | 2266 | 2510 |
| 13 | | | | 1406 | 1533 | | 1303 | 1591 | 1477 |
| 13 | | | | 1764 | 1632 | 647 | 1398 | 1573 | 1279 |
| 9 | 3995 | 4554 | 3770 | | | | | | |
| 9 | 3226 | 3325 | 4492 | | | | | | |
| 9 | 7080 | 4244 | 4465 | | | | | | |
| 9 | 4123 | 3978 | 3598 | | | | | | |
| 9 | 4485 | 4114 | 4600 | | | | | | |
| 11 | 4309 | 4913 | 4136 | | | | | | |
| 11 | 4044 | 5369 | 3666 | | | | | | |
| 11 | 6706 | 5637 | 5460 | 2650 | | | | | |
| 11 | 5518 | 5381 | 4740 | | | | | | |
| 11 | 4236 | 5493 | 4428 | | | | | | |

TABLE 5C
Detection of Stx2g, Stx2h, and Stx2i by Microarray.

| Probe | | | | | | | | | |
|---|---|---|---|---|---|---|---|---|---|
| SEQ | | | | | | | | | |
| ID NO | RFU for stx2i | | | RFU for stx2h | | | RFU for stx2g | | |
| 10 | 298 | 247 | 115 | 3195 | 3758 | 4009 | 2550 | 2620 | 2411 |
| 10 | 117 | 213 | 139 | 3071 | 4745 | 3873 | 3069 | 3226 | 3023 |
| 10 | 545 | 765 | | 4253 | 6454 | 5351 | 4226 | 3900 | 2422 |
| 10 | 127 | | 249 | 2560 | 4115 | 3764 | 2407 | 3391 | 2123 |
| 10 | | | | 2568 | 3277 | 3822 | 2403 | | 1771 |
| 8 | 1778 | 2532 | 1733 | 230 | 199 | 214 | 1212 | 1012 | 1173 |
| 8 | 3381 | 5145 | 2581 | | 1521 | 1075 | 1671 | 1454 | 1520 |
| 8 | 1737 | 2224 | 1468 | | | 209 | 972 | 917 | 996 |
| 8 | 1934 | 2161 | 1859 | 124 | | 192 | 1081 | 1375 | 984 |
| 8 | 2356 | 2379 | 2243 | 682 | 570 | 1015 | 1354 | 1335 | 1353 |
| 12 | 3155 | 4868 | 3049 | 648 | 370 | | 404 | 637 | 677 |
| 12 | 2975 | 3541 | 2315 | 366 | 343 | 525 | 447 | 283 | 361 |
| 12 | 2418 | 4138 | 2092 | 207 | 648 | 393 | 237 | 332 | 259 |
| 12 | 2144 | 3307 | 2486 | 279 | 281 | 533 | 385 | 342 | 228 |
| 12 | 4174 | 3926 | 2004 | 1536 | | 2000 | | | 253 |
| 7 | 961 | 1501 | 1037 | 908 | 1239 | | 1257 | 1109 | 1327 |
| 7 | 1273 | 1674 | 1510 | 1166 | 1540 | 1143 | 1283 | 1710 | 1487 |
| 7 | 1849 | | 2084 | 1744 | 2407 | 1857 | 2538 | 2226 | 2745 |
| 7 | 1365 | 1342 | 2813 | 877 | 1542 | 1304 | 1687 | 1715 | 1595 |
| 7 | 1278 | 1413 | 650 | 677 | 1026 | 970 | 1499 | 1353 | 1386 |
| 13 | 2302 | 3320 | 1956 | 1907 | 2734 | 2015 | 2550 | 2565 | 1258 |
| 13 | 1224 | 1389 | 1591 | 982 | 1457 | 1198 | 1566 | 1904 | 1481 |
| 13 | 3471 | | 1804 | 1332 | 2139 | 1525 | 2608 | 1892 | 1490 |
| 13 | 1104 | 1347 | 937 | 811 | 1177 | 1018 | 1402 | 1225 | 1392 |
| 13 | 1204 | 1299 | 889 | 924 | 1141 | 1059 | 1455 | 1218 | 1308 |
| 9 | | | 795 | | | | | | |
| 9 | | | | | | | | | |
| 9 | 2202 | 577 | | | | | | | |
| 9 | | | | | | | | | |
| 9 | | | | | | | | | |
| 11 | | | | | | | | | |
| 11 | | | | | | | | | |
| 11 | | | | | | | | | |
| 11 | | | 2192 | | | | | | |
| 11 | | | | | | | | | |

TABLE 5D
Detection of Stx2j and Stx2k by Microarray.

| Probe | | | | | | |
|---|---|---|---|---|---|---|
| SEQ ID NO | RFU for stx2j | | | RFU for stx2k | | |
| 10 | 2495 | 3255 | 3039 | 188 | 137 | 311 |
| 10 | 2547 | 3537 | 3436 | 160 | 145 | 407 |
| 10 | 6827 | 4233 | 4867 | | 839 | 344 |
| 10 | 3189 | 3051 | 2949 | 121 | | 257 |
| 10 | 3097 | 3493 | 4201 | | | 165 |
| 8 | | | | 1992 | 2611 | 2122 |
| 8 | | 1700 | 120 | 3445 | 2931 | 4640 |
| 8 | | | | 1739 | 2158 | 1739 |
| 8 | | | | 1955 | 3933 | 1902 |
| 8 | | 164 | | 2667 | 2671 | 2191 |
| 12 | 719 | 849 | 943 | 3674 | 3782 | 4414 |
| 12 | 480 | 844 | 850 | 2373 | 2758 | 3304 |
| 12 | 563 | 943 | 806 | 2682 | 2855 | 3271 |
| 12 | 460 | 799 | 721 | 3040 | 2900 | 3278 |
| 12 | 372 | | 1556 | 3394 | 3149 | 2499 |
| 7 | | | | 1085 | 1748 | 1056 |
| 7 | | | | 1705 | 1962 | 1305 |
| 7 | | | | 2174 | 2848 | 2193 |
| 7 | | | | 1896 | 1867 | 1457 |
| 7 | | | | 1340 | 1552 | 1070 |
| 13 | | | | 2716 | 2779 | 2389 |
| 13 | | | | 1494 | 2054 | 1031 |
| 13 | | | | 2005 | 2488 | 1564 |
| 13 | | | | 1179 | 1208 | 1144 |
| 13 | | | | 1181 | 1317 | 1181 |
| 9 | | | | | | |
| 9 | | | | 2123 | | |
| 9 | | 1814 | | | 2298 | |
| 9 | | | | | | |
| 9 | | | | | | |
| 11 | | | | | | |
| 11 | | | | | | |
| 11 | | | | | | |
| 11 | | | 658 | | | |
| 11 | | | | | | |

TABLE 6
Sequence Table - Exemplary Primers, Probes, and Templates for Amplification of stx2
Variants.

| SEQ | | | Target E. coli stx2 Variant(s) |
|---|---|---|---|
| ID | Sequence 5′ → 3′ | | (Exemplary Ref. |
| NO: | (Orientation) | Description | Seq./Nucleotide Positions) |
| 1 | TGGAGTTTAGTGGAAATGCC | Forward primer | stx2a, stx2b, stx2c, stx2d, stx2e, |
| | | | stx2f, stx2g, stx2h, stx2i, and |
| | | | stx2k |
| 2 | TGGAGTTCCGTGGTAATG | Forward primer | stx2a, stx2b, stx2c, stx2d, stx2e, |
| | | | stx2f, stx2g, stx2h, stx2i, and |
| | | | stx2k |
| 3 | TGGAGTTCAGTGGTAATACA | Forward primer | stx2a, stx2b, stx2c, stx2d, stx2e, |
| | | | stx2f, stx2g, stx2h, stx2i, and |
| | | | stx2k |
| 4 | TGGAGTTCAGAGGACGTTC | Forward primer | stx2a, stx2b, stx2c, stx2d, stx2e, |
| | | | stx2f, stx2g, stx2h, stx2i, and |
| | | | stx2k |
| 5 | TGCATCCAGAGCAGTTTTGC | Forward primer | stx2a, stx2b, stx2c, stx2d, stx2e, |
| | | | stx2f, stx2g, stx2h, stx2i, and |
| | | | stx2k |
| 6 | TGCATCCAGAGCAGTTCTGC | Forward primer | stx2a, stx2b, stx2c, stx2d, stx2e, |
| | | | stx2f, stx2g, stx2h, stx2i, and |
| | | | stx2k |
| 7 | TCTCTCTCTCTCTCTCTCTCTCACAGCAG | Probe | stx2e |
| | AAGCCTTACGGTT | | |
| 8 | TCTCTCTCTCTCTCTCTCTCGTCACAGCA | Probe | stx2a and stx2b |
| | GAAGCCTTACGC | | |
| 9 | TCTCTCTCTCTCTCTCTCTCGCGATTCAG | Probe | stx2f |
| | ACAAATACAGCGG | | |
| 10 | TCTCTCTCTCTCTCTCTCTCCGTTTCAGG | Probe | stx2a, stx2b, stx2c, stx2d, stx2e, |
| | CAAATACAGAGAG | | stx2g, stx2h, stx2i, stx2j, and |
| | | | stx2k |
| 11 | TCTCTCTCTCTCTCTCTCTCAGACAAATA | Probe | stx2f |
| | CAGCGGGGATTCC | | |
| 12 | TCTCTCTCTCTCTCTCTCTCACGCTTCAG | Probe | stx2a and stx2b |
| | GCAGATACAGAGA | | |
| 13 | TCTCTCTCTCTCTCTCTCTCACAGCAGAA | Probe | stx2e and stx2g |
| | GCCTTACGGTTCA | | |
| 14 | TCGTCATTCACTGGTTGG | Forward primer | stx2a, stx2b, stx2c, stx2d, stx2e, |
| | | | stx2f, stx2g, stx2h, stx2i, and |
| | | | stx 2k |
| 15 | TCGTCACTCACTGGTTTC | Forward primer | stx2a, stx2b, stx2c, stx2d, stx2e, |
| | | | stx2f, stx2g, stx2h, stx2i, and |
| | | | stx2k |
| 16 | TCACAGCAGAAGCCTTACGGTT | Probe | stx2e |
| 17 | TACGTTTCAGGCAAATACAGAGAGA | Probe | stx2b, stx2e, stx2g, stx2h, and |
| | | | stx 2j |
| 18 | GTCATTGTATAAAGCGGAGATG | Reverse primer | stx2a, stx2b, stx2c, stx2d, stx2e, |
| | | | stx2f, stx2g, stx2h, stx2i, and |
| | | | stx2k |
| 19 | GTCATTCACTGGTTGGTTC | Forward primer | stx2a, stx2b, stx2c, stx2d, stx2e, |
| | | | stx2f, stx2g, stx2h, stx2i, and |
| | | | stx 2k |
| 20 | GTCATCGTATAAACAGGAGC | Reverse primer | stx2a, stx2b, stx2c, stx2d, stx2e, |
| | | | stx2f, stx2g, stx2h, stx2i, and |
| | | | stx2k |
| 21 | GTCACTCACTGGTTTCATC | Forward primer | stx2a, stx2b, stx2c, stx2d, stx2e, |
| | | | stx2f, stx2g, stx2h, stx2i, and |
| | | | stx2k |
| 22 | GTCACAGCAGAAGCCTTACGC | Probe | stx2a and stx2b |
| 23 | GCTTCTAGGGCTGTCCTGC | Forward primer | stx2a, stx2b, stx2c, stx2d, stx2e, |
| | | | stx2f, stx2g, stx2h, stx2i, and |
| | | | stx2k |
| 24 | GCGGAGATGCCTCAGACAG | Reverse primer | stx2f |
| 25 | GCGATTCAGACAAATACAGCGG | Probe | stx2f |
| 26 | GACGCATCCAGAGCAGTTCT | Forward primer | stx2a, stx2b, stx2c, stx2d, stx2e, |
| | | | stx2f, stx2g, stx2h, stx2i, and |
| | | | stx2k |
| 27 | CTGGATTTAATGGAGTTCAGA | Forward primer | stx2a, stx2b, stx2c, stx2d, stx2e, |
| | | | stx2f, stx2g, stx2h, stx2i, and |
| | | | stx2k |
| 28 | CGTTTCAGGCAAATACAGAGAG | Probe | stx2a, stx2b, stx2c, stx2d, stx2e, |
| | | | stx2f, stx2g, stx2h, stx2i, and |
| | | | stx2k |
| 29 | CGTCCAGGGCTGTCCTGC | Forward primer | stx2a, stx2b, stx2c, stx2d, stx2e, |
| | | | stx2f, stx2g, stx2h, stx2i, and |
| | | | stx2k |
| 30 | CGTCATCGTATACACAGGAG | Reverse primer | stx2a, stx2b, stx2c, stx2d, stx2e, |
| | | | stx2f, stx2g, stx2h, stx2i, and |
| | | | stx2k |
| 31 | CATATCTGGCGTTAATGGAG | Forward primer | stx2a, stx2b, stx2c, stx2d, stx2e, |
| | | | stx2f, stx2g, stx2h, stx2i, and |
| | | | stx2k |
| 32 | CAGGCAGATACAGAGAGAATTTCGTCAG | Probe | stx2a, stx2b, and stx2d |
| | G | | |
| 33 | CAGGAGCTGCTTCAGATAGC | Reverse primer | stx2a, stx2b, stx2c, stx2d, stx2e, |
| | | | stx2f, stx2g, stx2h, stx2i, and |
| | | | stx2k |
| 34 | CAGCAGAAGCCTTACGGTTCAGGCAA | Probe | stx2e and stx2g |
| 35 | CAGACAAATACAGCGGGGATTCCGACC | Probe | stx2f |
| 36 | ATCTGGCGTTAATGGAGTT | Forward primer | stx2a, stx2b, stx2c, stx2d, stx2e, |
| | | | stx2f, stx2g, stx2h, stx2i, and |
| | | | stx2k |
| 37 | ATCATCCAGAGCTATGCTGCG | Forward primer | stx2f |
| 38 | AGACAAATACAGCGGGGATTCC | Probe | stx2f |
| 39 | ACGCTTCAGGCAGATACAGAGA | Probe | stx2a and stx2b |
| 40 | ACAGGAGCAGTTTCAGACGGT | Reverse primer | stx2a, stx2b, stx2c, stx2d, stx2e, |
| | | | stx2f, stx2g, stx2h, stx2i, and |
| | | | stx2k |
| 41 | ACAGGAGCAGTTTCAGACAGT | Reverse primer | stx2a and stx2b |
| 42 | ACAGCAGAAGCCTTACGGTTCA | Probe | stx2e and stx2g |
| 43 | NCMGGAGMWGYTTCAGAYRGY; | Reverse Primer | stx2 Primer Region |
| | N at position 1 is absent | Region | |
| | or adenine; when N is | | |
| | adenine, R is guanine. | | |
| 44 | CATATCTGGMKTTAATGGAGTTYMGWG | Primer | stx2 Primer Region |
| | GWMRTDCA | | |
| 45 | GTCGTCAYTCACTGGTTKSWTCA | Primer | stx2 Primer Region |
| 46 | CCCATGAGACATACAAAAAGGTAATGCC | Template | stx2a |
| | GCCGGCCTCGTAGTGAAAATTAATGGTG | | |
| | TTGAACAGATCGCGCACCAATACGGCGT | | |
| | CCTGCAGACAGTAACGGCCTCCGGAATG | | |
| | CAAATCAGTCGTCACTCACTGGTTTCAT | | |
| | CATATCTGGCGTTAATGGAGTTCAGTGG | | |
| | TAATACAATGACCAGAGATGCATCCAGA | | |
| | GCAGTTCTGCGTTTTGTCACTGTCACAGC | | |
| | AGAAGCCTTACGCTTCAGGCAGATACAG | | |
| | AGAGAATTTCGTCAGGCACTGTCTGAAA | | |
| | CTGCTCCTGTGTATACGATGACGCCGGG | | |
| | AGACGTGGACCTCACTGGTCTCGACTAT | | |
| | ACGCCCGTTTTCGGATC | | |
| 47 | CCCATGAGACATACAAAAAGGTAATGCC | Template | stx2b |
| | GCCGGCCTCGTAGTGAAAATTAATGGTG | | |
| | TTGAACAGATCGCGCACCAATACGGCGT | | |
| | CCTGCAGACAGTAACGGCCTCCGGAATG | | |
| | CAAATCAGTCGTCACTCACTGGTTTCAT | | |
| | CATATCTGGCGTTAATGGAGTTTAGTGG | | |
| | AAATGCCATGACCAGAGATGCATCCAGA | | |
| | GCAGTTCTGCGTTTTGTCACTGTCACAGC | | |
| | AGAAGCCTTACGGTTCAGGCAAATACAG | | |
| | AGAGAATTTCGTCTGGCACTGTCTGAAA | | |
| | CTGCTCCTGTTTATACGATGACACCGGA | | |
| | AGAAGTGGACCTCACAGGTCTCGACTAT | | |
| | ACGCCCGTTTTCGGATC | | |
| 48 | CCCATGAGACATACAAAAAGGTAATGCC | Template | stx2c |
| | GCCGGCCTCGTAGTGAAAATTAATGGTG | | |
| | TTGAACAGATCGCGCACCAATACGGCGT | | |
| | CCTGCAGACAGTAACGGCCTCCGGAATG | | |
| | CAAATCAGTCGTCACTCACTGGTTTCAT | | |
| | CATATCTGGCGTTAATGGAGTTCAGTGG | | |
| | TAATACAATGACCAGAGATGCATCCAGA | | |
| | GCAGTTCTGCGTTTTGTCACTGTCACAGC | | |
| | AGAAGCCTTACGCTTCAGGCAGATACAG | | |
| | AGAGAATTTCGTCAGGCACTGTCTGAAA | | |
| | CTGCTCCTGTGTATACGATGACGCCGGG | | |
| | AGACGTGGACCTCACTGGTCTCGACTAT | | |
| | ACGCCCGTTTTCGGATC | | |
| 49 | CCCATGAGACATACAAAAAGGTAATGCC | Template | stx2d |
| | GCCGGCCTCGTAGTGAAAATTAATGGTG | | |
| | TTGAACAGATCGCGCACCAATACGGCGT | | |
| | CCTGCAGACAGTAACGGCCTTCCGGAAT | | |
| | GCAAATCAGTCGTCACTCACTGGTTTCA | | |
| | TCATATCTGGCGTTAATGGAGTTCAGTG | | |
| | GTAATACAATGACCAGAGATGCATCCAG | | |
| | AGCAGTTCTGCGTTTTGTCACTGTCACA | | |
| | GCAGAAGCCTTACGCTTCAGGCAGATAC | | |
| | AGAGAGAATTTCGTCAGGCACTGTCTGA | | |
| | AACTGCTCCTGTGTATACGATGACGCCG | | |
| | GGAGACGTGGACCTCACTGGTCTCGACT | | |
| | ATACGCCCGTTTTCGGATC | | |
| 50 | CCCATGAGACATACAAAAAGGTAATGCC | Template | stx2e |
| | GCCGGCCTCGTAGTGAAAATTAATGGTG | | |
| | TTGAACAGATCGCGCACCAATACGGCGT | | |
| | CCTGCAGACAGTAACGGCCTCCGGAATG | | |
| | CAAATCAGTCGTCACTCACTGGTTTCAT | | |
| | CATATCTGGCGTTAATGGAGTTCAGTGG | | |
| | TAATACAATGACCAGAGATGCATCAAGA | | |
| | GCAGTTCTGCGTTTTGTCACTGTCACAGC | | |
| | AGAAGCCTTACGGTTCAGGCAAATACAG | | |
| | AGAGAATTTCGTCTGGCACTGTCTGAAA | | |
| | CTGCTCCTGTTTATACGATGACGCCGGA | | |
| | AGACGTGGACCTCACTGGTCTCGACTAT | | |
| | ACGCCCGTTTTCGGATC | | |
| 51 | CCCATGAGACATACAAAAAGGTAATGCC | Template | stx2f |
| | GCCGGCCTCGTAGTGAAAATTAATGGTG | | |
| | TTGAACAGATCGCGCACCAATACGGCGT | | |
| | CCTGCAGACAGTAACGGCCACAGGGAT | | |
| | GCAGATTGGGCGTCATTCACTGGTTGGT | | |
| | TCATATCTGGATTTAATGGAGTTCAGAG | | |
| | GACGTTCCATGACCCGCGCATCATCCAG | | |
| | AGCTATGCTGCGTTTTGTCACAGTGATA | | |
| | GCAGAAGCTCTGCGATTCAGACAAATAC | | |
| | AGCGGGGATTCCGACCGGCGCTGTCTGA | | |
| | GGCATCTCCGCTTTATACAATGACGGCT | | |
| | CAGGATGTTGACCTTACCGGTCTCGACT | | |
| | ATACGCCCGTTTTCGGATC | | |
| 52 | CCCATGAGACATACAAAAAGGTAATGCC | Template | stx2g |
| | GCCGGCCTCGTAGTGAAAATTAATGGTG | | |
| | TTGAACAGATCGCGCACCAATACGGCGT | | |
| | CCTGCAGACAGTAACGGCCTCCGGAATG | | |
| | CAAATCAGTCGTCACTCACTGGTTTCAT | | |
| | CATATCTGGCGTTAATGGAGTTCAGTGG | | |
| | TAATACAATGACCAGAGACGCATCCAGA | | |
| | GCAGTTCTTCGTTTTGTCACTGTTACAGC | | |
| | AGAAGCCTTACGGTTCAGGCAAATACAG | | |
| | AGAGAATTTCGTCTGGCACTGTCTGAAA | | |
| | CTGCTCCTGTTTATACGATGACGCCGGA | | |
| | AGACGTGGACCTCACTGGTCTCGACTAT | | |
| | ACGCCCGTTTTCGGATC | | |
| 53 | CCCATGAGACATACAAAAAGGTAATGCC | Template | stx2h |
| | GCCGGCCTCGTAGTGAAAATTAATGGTG | | |
| | TTGAACAGATCGCGCACCAATACGGCGT | | |
| | CCTGCAGACAGTAACGGCCTCCGGAATG | | |
| | CAAATCAGTCGTCACTCACTGGTTTCAT | | |
| | CATATCTGGCGTTAATGGAGTTCAGTGG | | |
| | TAATGCCATGACCAGAGAGGCTTCCAGG | | |
| | GCTGTCCTGCGTTTTGTCACTGTTACAGC | | |
| | CGAAGCCTTACGGTTCAGGCAAATACAG | | |
| | AGAGAATTTCGTCAGGCGCTATCTGAAG | | |
| | CAGCTCCTGTTTATACGATGACACCGGA | | |
| | AGACGTGGACCTTACTGGTCTCGACTAT | | |
| | ACGCCCGTTTTCGGATC | | |
| 54 | CCCATGAGACATACAAAAAGGTAATGCC | Template | stx2i |
| | GCCGGCCTCGTAGTGAAAATTAATGGTG | | |
| | TTGAACAGATCGCGCACCAATACGGCGT | | |
| | CCTGCAGACAGTAACGGCCTTCCGGAAT | | |
| | GCAAATCAGTCGTCACTCACTGGTTTCA | | |
| | TCATATCTGGCGTTAATGGAGTTCAGTG | | |
| | GTAATACAATGACCAGAGAGGCTTCTAG | | |
| | GGCTGTCCTGCGTTTTGTAACCGTCACA | | |
| | GCCGAAGCCTTACGGTTCAGGCAGATAC | | |
| | AGAGAGAATTTCGTCAGGCACTGTCTGA | | |
| | AACAGCTCCTGTTTATACGATGACGCCG | | |
| | GAAGACGTGGACCTCACTGGTCTCGACT | | |
| | ATACGCCCGTTTTCGGATC | | |
| 55 | CCCATGAGACATACAAAAAGGTAATGCC | Template | stx2j |
| | GCCGGCCTCGTAGTGAAAATTAATGGTG | | |
| | TTGAACAGATCGCGCACCAATACGGCGT | | |
| | CCTGCAGACAGTAACGGCCCGCAGCGCT | | |
| | GGAACGCTCCGGAATGCAAATCAGCCGT | | |
| | CACTCACTGGTTTCATCATATCTGGAGTT | | |
| | AATGGAGTTCCGTGGTAATGCCATGACC | | |
| | AGAGCGGCGTCCAGGGCTGTCCTGCGTT | | |
| | TTGTCACTGTCACAGCCGAGGCTTTACG | | |
| | TTTCAGGCAAATACAGAGAGAATTTCGT | | |
| | CCGGCGCTATCTGAAGCTGCTCCTGTTT | | |
| | ATACGATGACACCGGAAGACGTGGACCT | | |
| | CACTCTGAACTGGGGGAGAATCGGTCTC | | |
| | GACTATACGCCCGTTTTCGGATC | | |
| 56 | CCCATGAGACATACAAAAAGGTAATGCC | Template | stx2k |
| | GCCGGCCTCGTAGTGAAAATTAATGGTG | | |
| | TTGAACAGATCGCGCACCAATACGGCGT | | |
| | CCTGCAGACAGTAACGGCCATCAGTCGT | | |
| | CACTCACTGGTTTCATCATATCTGGCGTT | | |
| | AATGGAGTTCAGTGGTAATACAATGACC | | |
| | AGAGAGGCTTCTAGGGCTGTCCTGCGTT | | |
| | TTGTAACCGTCACAGCCGAAGCCTTACG | | |
| | GTTCAGGCAGATACAGAGAGAATTTCGT | | |
| | CAGGCACTGTCTGAAACAGCTCCTGTTT | | |
| | ATACGATGACGCCGGAAGACGTGGACCT | | |
| | GGTCTCGACTATACGCCCGTTTTCGGAT | | |
| | C | | |
| 57 | CCCATGAGACATACAAAAAGGTAATGCC | Template | stx21 |
| | GCCGGCCTCGTAGTGAAAATTAATGGTG | | |
| | TTGAACAGATCGCGCACCAATACGGCGT | | |
| | CCTGCAGACAGTAACGGCCTCAGTGGTA | | |
| | ATACAATGACCAGAGATGCATCCAGAGC | | |
| | AGTTCTGCGTTTTGTCACTGTCACAGCA | | |
| | GAAGCCTTACGCTTCAGGCAGATACAGA | | |
| | GAGAATTTCGTCAGGCACCGTCTGAAAC | | |
| | TGCTCCTGTGTGCATACGATGACGCCGG | | |
| | GAGACGTGGAGGTCTCGACTATACGCCC | | |
| | GTTTTCGGATC | | |
| 58 | CCCATGAGACATACAAAAAGGTAATGCC | Template | Unspecified stx2 variant |
| | GCCGGCCTCGTAGTGAAAATTAATGGTG | | |
| | TTGAACAGATCGCGCACCAATACGGCGT | | |
| | CCTGCAGACAGTAACGGCCTTAGTGGAA | | |
| | ATGCCATGACCAGAGATGCATCCAGAGC | | |
| | AGTTTTGCGTTTTGTCACTGTCACAGCAG | | |
| | AAGCCTTACGGTTCAGGCAAATACAGAG | | |
| | AGAATTTCGTCTGGCACTGTCTGAAACT | | |
| | GCTCCTGTTTATACGATGACACCGGAAG | | |
| | AAGTGGGGTCTCGACTATACGCCCGTTT | | |
| | TCGGATC | | |
| 59 | TCTCTCTCTCTCTCTCTCTCCTCTCTGT | Microarray probe | |
| | ATTTGCCTGAAACG | | |
| 60 | TCTCTCTCTCTCTCTCTCTCTACGTTT | Microarray probe | |
| | CAGGCAAATACAGAGAGA | | |
| 61 | TCTCTCTCTCTCTCTCTCTCTCTCTCT | Microarray probe | |
| | GTATTTGCCTGAAACGTA | | |
| 62 | CTCTCTGTATTTGCCTGAAACG | Microarray probe | |
| 63 | TCTCTCTGTATTTGCCTGAAACGTA | Microarray probe | |

From the foregoing, it will be appreciated that, although specific embodiments of the invention have been described herein for purposes of illustration, various modifications may be made without deviating from the spirit and scope of the invention. Accordingly, the invention is not limited except as by the appended claims. All publications, patents, and patent applications cited herein are hereby incorporated by reference in their entireties for all purposes.

1. A multiplex method for determining the presence or absence one or more variants of a stx target nucleic acid in a sample, said method comprising:
a. contacting a sample, said sample suspected of containing a stx target nucleic acid with a plurality of stx-specific amplification oligomers for amplifying a target region of the stx target nucleic acid, wherein:
i. the plurality of stx-specific amplification oligomers comprises first, second, third, fourth, fifth, and sixth oligomers respectively comprising target-hybridizing sequences consisting of the nucleotide sequences of: (i) SEQ ID NO: 29; (ii) SEQ ID NO: 5; (iii) SEQ ID NO: 23; (iv) SEQ ID NO: 26; (v) SEQ ID NO: 33; and (vi) SEQ ID NO: 40;
ii. the plurality of stx-specific amplification oligomers comprises first, second, third, fourth, and fifth oligomers respectively comprising target-hybridizing sequences consisting of the nucleotide sequences of: (i) SEQ ID NO: 14; (ii) SEQ ID NO: 15; (iii) SEQ ID NO: 18; (iv) SEQ ID NO: 30; and (v) SEQ ID NO: 20;
iii. the plurality of stx-specific amplification oligomers comprises first, second, third, fourth, and fifth oligomers respectively comprising target-hybridizing sequences consisting of the nucleotide sequences of: (i) SEQ ID NO: 21; (ii) SEQ ID NO: 19; (iii) SEQ ID NO: 18; (iv) SEQ ID NO: 30; and (v) SEQ ID NO: 20;
iv. the plurality of stx-specific amplification oligomers comprises first, second, third, fourth, and fifth oligomers respectively comprising target-hybridizing sequences consisting of the nucleotide sequences of: (i) SEQ ID NO: 31 and; (ii) SEQ ID NO: 27; (iii) SEQ ID NO: 18; (iv) SEQ ID NO: 30; and (v) SEQ ID NO: 20;
v. the plurality of stx-specific amplification oligomers comprises first, second, third, fourth, and fifth oligomers respectively comprising target-hybridizing sequences consisting of the nucleotide sequences of: (i) SEQ ID NO: 36; (ii) SEQ ID NO: 27; (iii) SEQ ID NO: 18; (iv) SEQ ID NO: 30; and (v) SEQ ID NO: 20; or
vi. the plurality of stx-specific amplification oligomers comprises first, second, third, fourth, fifth, sixth, and seventh oligomers respectively comprising target-hybridizing sequences consisting of the nucleotide sequences of: (i) SEQ ID NO: 3; (ii) SEQ ID NO: 4; (iii) SEQ ID NO: 18; (iv) SEQ ID NO: 30; (v) SEQ ID NO: 20; (vi) SEQ ID NO: 1; and (vii) SEQ ID NO: 2;
b. performing an in vitro nucleic acid amplification reaction, wherein the stx target nucleic acid, if present in said sample, is used as a template for generating one or more amplification products corresponding to the stx target regions; and
c. (i) determining the sequences of the one or more amplification products, or (ii) detecting the presence or absence of the one or more amplification products using a plurality of detection probes, wherein the detection probes are specific for one or more variants of the stx target nucleic acid,
thereby determining the presence or absence of the one or more variants of the stx target nucleic acid in said sample.

2. The method of claim 1, wherein the plurality of stx-specific amplification oligomers further comprises first, second, third, and fourth additional stx-specific amplification oligomers for amplifying the target region of the stx target nucleic acid, wherein the first, second, third, and fourth additional stx-specific amplification oligomers respectively comprise first, second, third, and fourth target-hybridizing sequences consisting of the nucleotide sequences of: (i) SEQ ID NO: 37; (ii) SEQ ID NO: 6; (iii) SEQ ID NO: 24; and (iv) SEQ ID NO: 41.

3. The method of claim 1, wherein the plurality of detection probes comprises (i) a first detection probe comprising a target-hybridizing sequence consisting of the nucleotide sequence of SEQ ID NO: 28 or 17; or (ii) a first detection probe comprising a target-hybridizing sequence consisting of the nucleotide sequence of SEQ ID NO: 28 and an additional detection probe comprising a target-hybridizing sequence consisting of the nucleotide sequence of SEQ ID NO: 17.

4. The method of claim 3, wherein the first detection probe comprises the nucleotide sequence of SEQ ID NO: 10.

5. The method of claim 3, wherein the plurality of detection probes comprises a second detection probe comprising a target-hybridizing sequence consisting of the nucleotide sequence of SEQ ID NO: 22; a third detection probe comprising a target-hybridizing sequence consisting of the nucleotide sequence of SEQ ID NO: 39; a fourth detection probe comprising a target-hybridizing sequence consisting of the nucleotide sequence of SEQ ID NO: 16; a fifth detection probe comprising a target-hybridizing sequence consisting of the nucleotide sequence of SEQ ID NO: 42; a sixth detection probe comprising a target-hybridizing sequence consisting of the nucleotide sequence of SEQ ID NO: 38; and/or a seventh detection probe comprising a target-hybridizing sequence consisting of the nucleotide sequence of SEQ ID NO: 25.

6. The method of claim 5, wherein the second detection probe comprises the nucleotide sequence of SEQ ID NO: 8; the third detection probe comprises the nucleotide sequence of SEQ ID NO: 12; the fourth detection probe comprises the nucleotide sequence of SEQ ID NO: 7; the fifth detection probe comprises the nucleotide sequence of SEQ ID NO: 13; the sixth detection probe comprises the nucleotide sequence of SEQ ID NO: 11; and/or the seventh detection probe comprises the nucleotide sequence of SEQ ID NO: 9.

7-31. (canceled)

32. The method of claim 1, wherein presence or absence of the one or more amplification products are detected using the plurality of detection probes.

33. The method of claim 32, wherein each of the plurality of detection probes comprises a fluorescent label, optionally wherein each of the plurality of detection probes further comprises a quenching label.

34. (canceled)

35. The method of claim 32, wherein each of the plurality of detection probes is immobilized on a solid support.

36. The method of claim 35, wherein the solid support is a microarray and detection comprises hybridization of amplified products to one or more probes immobilized on the microarray.

37. The method of claim 1, wherein one or more of the stx-specific amplification oligomers comprises a fluorescent label.

38-41. (canceled)

42. A set of oligonucleotides for determining the presence or absence one or more variants of a stx target nucleic acid in a sample, said oligonucleotide set comprising a plurality of stx-specific amplification oligomers for amplifying a target region of the stx target nucleic acid, wherein:
a. the plurality of stx-specific amplification oligomers comprises first, second, third, fourth, fifth, and sixth oligomers respectively comprising target-hybridizing sequences consisting of the nucleotide sequences of: (i) SEQ ID NO: 29; (ii) SEQ ID NO: 5; (iii) SEQ ID NO: 23; (iv) SEQ ID NO: 26; (v) SEQ ID NO: 33; and (vi) SEQ ID NO: 40;
b. the plurality of stx-specific amplification oligomers comprises first, second, third, fourth, and fifth oligomers respectively comprising target-hybridizing sequences consisting of the nucleotide sequences of: (i) SEQ ID NO: 14; (ii) SEQ ID NO: 15; (iii) SEQ ID NO: 18; (iv) SEQ ID NO: 30; and (v) SEQ ID NO: 20;
c. the plurality of stx-specific amplification oligomers comprises first, second, third, fourth, and fifth oligomers respectively comprising target-hybridizing sequences consisting of the nucleotide sequences of: (i) SEQ ID NO: 21; (ii) SEQ ID NO: 19; (iii) SEQ ID NO: 18; (iv) SEQ ID NO: 30; and (v) SEQ ID NO: 20;
d. the plurality of stx-specific amplification oligomers comprises first, second, third, fourth, and fifth oligomers respectively comprising target-hybridizing sequences consisting of the nucleotide sequences of: (i) SEQ ID NO: 31 and; (ii) SEQ ID NO: 27; (iii) SEQ ID NO: 18; (iv) SEQ ID NO: 30; and (v) SEQ ID NO: 20;
e. the plurality of stx-specific amplification oligomers comprises first, second, third, fourth, and fifth oligomers respectively comprising target-hybridizing sequences consisting of the nucleotide sequences of: (i) SEQ ID NO: 36; (ii) SEQ ID NO: 27; (iii) SEQ ID NO: 18; (iv) SEQ ID NO: 30; and (v) SEQ ID NO: 20; or
f. the plurality of stx-specific amplification oligomers comprises first, second, third, fourth, fifth, sixth, and seventh oligomers respectively comprising target-hybridizing sequences consisting of the nucleotide sequences of: (i) SEQ ID NO: 3; (ii) SEQ ID NO: 4; (iii) SEQ ID NO: 18; (iv) SEQ ID NO: 30; (v) SEQ ID NO: 20; (vi) SEQ ID NO: 1; and (vii) SEQ ID NO: 2.

43. The oligonucleotide set of claim 42, wherein the plurality of stx-specific amplification oligomers further comprises first, second, third, and fourth additional stx-specific amplification oligomers for amplifying the target region of the stx target nucleic acid, wherein the first, second, third, and fourth additional stx-specific amplification oligomers respectively comprise first, second, third, and fourth target-hybridizing sequences consisting of the nucleotide sequences of: (i) SEQ ID NO: 37; (ii) SEQ ID NO: 6; (iii) SEQ ID NO: 24; and (iv) SEQ ID NO: 41.

44-59. (canceled)

60. The oligonucleotide set of claim 42, further comprising a plurality of detection probes, wherein the plurality of detection probes comprises a first detection probe comprising a target-hybridizing sequence consisting of the nucleotide sequence of SEQ ID NO: 28 or 17; a second detection probe comprising a target-hybridizing sequence consisting of the nucleotide sequence of SEQ ID NO: 22; a third detection probe comprising a target-hybridizing sequence consisting of the nucleotide sequence of SEQ ID NO: 39; a fourth detection probe comprising a target-hybridizing sequence consisting of the nucleotide sequence of SEQ ID NO: 16; a fifth detection probe comprising a target-hybridizing sequence consisting of the nucleotide sequence of SEQ ID NO: 42; a sixth detection probe comprising a target-hybridizing sequence consisting of the nucleotide sequence of SEQ ID NO: 38; and a seventh detection probe comprising a target-hybridizing sequence consisting of the nucleotide sequence of SEQ ID NO: 25.

61-63. (canceled)

64. The oligonucleotide set of claim 60, wherein each of the plurality of detection probes is immobilized on a solid support, optionally wherein the solid support is a microarray.

65. (canceled)

66. The oligonucleotide set of claim 60, wherein each of the plurality of detection probes comprises a fluorescent label, optionally wherein each of the plurality of detection probes further comprises a non-fluorescent quenching label.

67. (canceled)

68. The oligonucleotide set of claim 42, wherein one or more of the stx-specific amplification oligomers comprises a fluorescent label.

69. The oligonucleotide set of claim 68, wherein two or more of the stx-specific amplification oligomers comprise a fluorescent label so that each amplification product corresponding to a stx target region comprises a fluorescent label incorporated from a stx-specific amplification oligomer.

70. (canceled)

71. (canceled)

72. A kit comprising the oligonucleotide set of claim 42.

73. A reaction mixture comprising the oligonucleotide set of claim 42.


SEQUENCE LISTING (63 sequences)

SEQ ID NO: 1 - DNA, 20 bp, synthetic construct
  misc_feature 1..20: note = Forward primer
  source 1..20: mol_type = other DNA
tggagtttagtggaaatgcc

SEQ ID NO: 2 - DNA, 18 bp, synthetic construct
  misc_feature 1..18: note = Forward primer
  source 1..18: mol_type = other DNA
tggagttccgtggtaatg

SEQ ID NO: 3 - DNA, 20 bp, synthetic construct
  misc_feature 1..20: note = Forward primer
  source 1..20: mol_type = other DNA
tggagttcagtggtaataca

SEQ ID NO: 4 - DNA, 19 bp, synthetic construct
  misc_feature 1..19: note = Forward primer
  source 1..19: mol_type = other DNA
tggagttcagaggacgttc

SEQ ID NO: 5 - DNA, 20 bp, synthetic construct
  misc_feature 1..20: note = Forward primer
  source 1..20: mol_type = other DNA
tgcatccagagcagttttgc

SEQ ID NO: 6 - DNA, 20 bp, synthetic construct
  misc_feature 1..20: note = Forward primer
  source 1..20: mol_type = other DNA
tgcatccagagcagttctgc

SEQ ID NO: 7 - DNA, 42 bp, synthetic construct
  misc_feature 1..42: note = Probe
  source 1..42: mol_type = other DNA
tctctctctctctctctctctcacagcagaagccttacggtt

SEQ ID NO: 8 - DNA, 41 bp, synthetic construct
  misc_feature 1..41: note = Probe
  source 1..41: mol_type = other DNA
tctctctctctctctctctcgtcacagcagaagccttacgc

SEQ ID NO: 9 - DNA, 42 bp, synthetic construct
  misc_feature 1..42: note = Probe
  source 1..42: mol_type = other DNA
tctctctctctctctctctcgcgattcagacaaatacagcgg

SEQ ID NO: 10 - DNA, 42 bp, synthetic construct
  misc_feature 1..42: note = Probe
  source 1..42: mol_type = other DNA
tctctctctctctctctctccgtttcaggcaaatacagagag

SEQ ID NO: 11 - DNA, 42 bp, synthetic construct
  misc_feature 1..42: note = Probe
  source 1..42: mol_type = other DNA
tctctctctctctctctctcagacaaatacagcggggattcc

SEQ ID NO: 12 - DNA, 42 bp, synthetic construct
  misc_feature 1..42: note = Probe
  source 1..42: mol_type = other DNA
tctctctctctctctctctcacgcttcaggcagatacagaga

SEQ ID NO: 13 - DNA, 42 bp, synthetic construct
  misc_feature 1..42: note = Probe
  source 1..42: mol_type = other DNA
tctctctctctctctctctcacagcagaagccttacggttca

SEQ ID NO: 14 - DNA, 18 bp, synthetic construct
  misc_feature 1..18: note = Forward primer
  source 1..18: mol_type = other DNA
tcgtcattcactggttgg

SEQ ID NO: 15 - DNA, 18 bp, synthetic construct
  misc_feature 1..18: note = Forward primer
  source 1..18: mol_type = other DNA
tcgtcactcactggtttc

SEQ ID NO: 16 - DNA, 22 bp, synthetic construct
  misc_feature 1..22: note = Probe
  source 1..22: mol_type = other DNA
tcacagcagaagccttacggtt

SEQ ID NO: 17 - DNA, 25 bp, synthetic construct
  misc_feature 1..25: note = Probe
  source 1..25: mol_type = other DNA
tacgtttcaggcaaatacagagaga

SEQ ID NO: 18 - DNA, 22 bp, synthetic construct
  misc_feature 1..22: note = Reverse primer
  source 1..22: mol_type = other DNA
gtcattgtataaagcggagatg

SEQ ID NO: 19 - DNA, 19 bp, synthetic construct
  misc_feature 1..19: note = Forward primer
  source 1..19: mol_type = other DNA
gtcattcactggttggttc

SEQ ID NO: 20 - DNA, 20 bp, synthetic construct
  misc_feature 1..20: note = Reverse primer
  source 1..20: mol_type = other DNA
gtcatcgtataaacaggagc

SEQ ID NO: 21 - DNA, 19 bp, synthetic construct
  misc_feature 1..19: note = Forward primer
  source 1..19: mol_type = other DNA
gtcactcactggtttcatc

SEQ ID NO: 22 - DNA, 21 bp, synthetic construct
  misc_feature 1..21: note = Probe
  source 1..21: mol_type = other DNA
gtcacagcagaagccttacgc

SEQ ID NO: 23 - DNA, 19 bp, synthetic construct
  misc_feature 1..19: note = Forward primer
  source 1..19: mol_type = other DNA
gcttctagggctgtcctgc

SEQ ID NO: 24 - DNA, 19 bp, synthetic construct
  misc_feature 1..19: note = Reverse primer
  source 1..19: mol_type = other DNA
gcggagatgcctcagacag

SEQ ID NO: 25 - DNA, 22 bp, synthetic construct
  misc_feature 1..22: note = Probe
  source 1..22: mol_type = other DNA
gcgattcagacaaatacagcgg

SEQ ID NO: 26 - DNA, 20 bp, synthetic construct
  misc_feature 1..20: note = Forward primer
  source 1..20: mol_type = other DNA
gacgcatccagagcagttct

SEQ ID NO: 27 - DNA, 21 bp, synthetic construct
  misc_feature 1..21: note = Forward primer
  source 1..21: mol_type = other DNA
ctggatttaatggagttcaga

SEQ ID NO: 28 - DNA, 22 bp, synthetic construct
  misc_feature 1..22: note = Probe
  source 1..22: mol_type = other DNA
cgtttcaggcaaatacagagag

SEQ ID NO: 29 - DNA, 18 bp, synthetic construct
  misc_feature 1..18: note = Forward primer
  source 1..18: mol_type = other DNA
cgtccagggctgtcctgc

SEQ ID NO: 30 - DNA, 20 bp, synthetic construct
  misc_feature 1..20: note = Reverse primer
  source 1..20: mol_type = other DNA
cgtcatcgtatacacaggag

SEQ ID NO: 31 - DNA, 20 bp, synthetic construct
  misc_feature 1..20: note = Forward primer
  source 1..20: mol_type = other DNA
catatctggcgttaatggag

SEQ ID NO: 32 - DNA, 29 bp, synthetic construct
  misc_feature 1..29: note = Probe
  source 1..29: mol_type = other DNA
caggcagatacagagagaatttcgtcagg

SEQ ID NO: 33 - DNA, 20 bp, synthetic construct
  misc_feature 1..20: note = Reverse primer
  source 1..20: mol_type = other DNA
caggagctgcttcagatagc

SEQ ID NO: 34 - DNA, 26 bp, synthetic construct
  misc_feature 1..26: note = Probe
  source 1..26: mol_type = other DNA
cagcagaagccttacggttcaggcaa

SEQ ID NO: 35 - DNA, 27 bp, synthetic construct
  misc_feature 1..27: note = Probe
  source 1..27: mol_type = other DNA
cagacaaatacagcggggattccgacc

SEQ ID NO: 36 - DNA, 19 bp, synthetic construct
  misc_feature 1..19: note = Forward primer
  source 1..19: mol_type = other DNA
atctggcgttaatggagtt

SEQ ID NO: 37 - DNA, 21 bp, synthetic construct
  misc_feature 1..21: note = Forward primer
  source 1..21: mol_type = other DNA
atcatccagagctatgctgcg

SEQ ID NO: 38 - DNA, 22 bp, synthetic construct
  misc_feature 1..22: note = Probe
  source 1..22: mol_type = other DNA
agacaaatacagcggggattcc

SEQ ID NO: 39 - DNA, 22 bp, synthetic construct
  misc_feature 1..22: note = Probe
  source 1..22: mol_type = other DNA
acgcttcaggcagatacagaga

SEQ ID NO: 40 - DNA, 21 bp, synthetic construct
  misc_feature 1..21: note = Reverse primer
  source 1..21: mol_type = other DNA
acaggagcagtttcagacggt

SEQ ID NO: 41 - DNA, 21 bp, synthetic construct
  misc_feature 1..21: note = Reverse primer
  source 1..21: mol_type = other DNA
acaggagcagtttcagacagt

SEQ ID NO: 42 - DNA, 22 bp, synthetic construct
  misc_feature 1..22: note = Probe
  source 1..22: mol_type = other DNA
acagcagaagccttacggttca

SEQ ID NO: 43 - DNA, 21 bp, synthetic construct
  misc_feature 1..21: note = Forward primer
  misc_feature 1: note = n is any nucleotide
  source 1..21: mol_type = other DNA
ncmggagmwgyttcagayrgy

SEQ ID NO: 44 - DNA, 35 bp, synthetic construct
  misc_feature 1..35: note = Primer
  source 1..35: mol_type = other DNA
catatctggmkttaatggagttymgwggwmrtdca

SEQ ID NO: 45 - DNA, 23 bp, synthetic construct
  misc_feature 1..23: note = Primer
  source 1..23: mol_type = other DNA
gtcgtcaytcactggttkswtca

SEQ ID NO: 46 - DNA, 354 bp, synthetic construct
  misc_feature 1..354: note = Template
  source 1..354: mol_type = other DNA
cccatgagacatacaaaaaggtaatgccgccggcctcgtagtgaaaattaatggtgttgaacagatcgcgcaccaatacggcgtcctgcagacagtaacggcctccggaatgcaaatcagtcgtcactcactggtttcatcatatctggcgttaatggagttcagtggtaatacaatgaccagagatgcatccagagcagttctgcgttttgtcactgtcacagcagaagccttacgcttcaggcagatacagagagaatttcgtcaggcactgtctgaaactgctcctgtgtatacgatgacgccgggagacgtggacctcactggtctcgactatacgcccgttttcggatc

SEQ ID NO: 47 - DNA, 354 bp, synthetic construct
  misc_feature 1..354: note = Template
  source 1..354: mol_type = other DNA
cccatgagacatacaaaaaggtaatgccgccggcctcgtagtgaaaattaatggtgttgaacagatcgcgcaccaatacggcgtcctgcagacagtaacggcctccggaatgcaaatcagtcgtcactcactggtttcatcatatctggcgttaatggagtttagtggaaatgccatgaccagagatgcatccagagcagttctgcgttttgtcactgtcacagcagaagccttacggttcaggcaaatacagagagaatttcgtctggcactgtctgaaactgctcctgtttatacgatgacaccggaagaagtggacctcacaggtctcgactatacgcccgttttcggatc

SEQ ID NO: 48 - DNA, 354 bp, synthetic construct; identical to SEQ ID NO: 46
  misc_feature 1..354: note = Template
  source 1..354: mol_type = other DNA

SEQ ID NO: 49 - DNA, 355 bp, synthetic construct
  misc_feature 1..355: note = Template
  source 1..355: mol_type = other DNA
cccatgagacatacaaaaaggtaatgccgccggcctcgtagtgaaaattaatggtgttgaacagatcgcgcaccaatacggcgtcctgcagacagtaacggccttccggaatgcaaatcagtcgtcactcactggtttcatcatatctggcgttaatggagttcagtggtaatacaatgaccagagatgcatccagagcagttctgcgttttgtcactgtcacagcagaagccttacgcttcaggcagatacagagagaatttcgtcaggcactgtctgaaactgctcctgtgtatacgatgacgccgggagacgtggacctcactggtctcgactatacgcccgttttcggatc

SEQ ID NO: 50 - DNA, 354 bp, synthetic construct
  misc_feature 1..354: note = Template
  source 1..354: mol_type = other DNA
cccatgagacatacaaaaaggtaatgccgccggcctcgtagtgaaaattaatggtgttgaacagatcgcgcaccaatacggcgtcctgcagacagtaacggcctccggaatgcaaatcagtcgtcactcactggtttcatcatatctggcgttaatggagttcagtggtaatacaatgaccagagatgcatcaagagcagttctgcgttttgtcactgtcacagcagaagccttacggttcaggcaaatacagagagaatttcgtctggcactgtctgaaactgctcctgtttatacgatgacgccggaagacgtggacctcactggtctcgactatacgcccgttttcggatc

SEQ ID NO: 51 - DNA, 354 bp, synthetic construct
  misc_feature 1..354: note = Template
  source 1..354: mol_type = other DNA
cccatgagacatacaaaaaggtaatgccgccggcctcgtagtgaaaattaatggtgttgaacagatcgcgcaccaatacggcgtcctgcagacagtaacggccacagggatgcagattgggcgtcattcactggttggttcatatctggatttaatggagttcagaggacgttccatgacccgcgcatcatccagagctatgctgcgttttgtcacagtgatagcagaagctctgcgattcagacaaatacagcggggattccgaccggcgctgtctgaggcatctccgctttatacaatgacggctcaggatgttgaccttaccggtctcgactatacgcccgttttcggatc

SEQ ID NO: 52 - DNA, 354 bp, synthetic construct
  misc_feature 1..354: note = Template
  source 1..354: mol_type = other DNA
cccatgagacatacaaaaaggtaatgccgccggcctcgtagtgaaaattaatggtgttgaacagatcgcgcaccaatacggcgtcctgcagacagtaacggcctccggaatgcaaatcagtcgtcactcactggtttcatcatatctggcgttaatggagttcagtggtaatacaatgaccagagacgcatccagagcagttcttcgttttgtcactgttacagcagaagccttacggttcaggcaaatacagagagaatttcgtctggcactgtctgaaactgctcctgtttatacgatgacgccggaagacgtggacctcactggtctcgactatacgcccgttttcggatc

SEQ ID NO: 53 - DNA, 354 bp, synthetic construct
  misc_feature 1..354: note = Template
  source 1..354: mol_type = other DNA
cccatgagacatacaaaaaggtaatgccgccggcctcgtagtgaaaattaatggtgttgaacagatcgcgcaccaatacggcgtcctgcagacagtaacggcctccggaatgcaaatcagtcgtcactcactggtttcatcatatctggcgttaatggagttcagtggtaatgccatgaccagagaggcttccagggctgtcctgcgttttgtcactgttacagccgaagccttacggttcaggcaaatacagagagaatttcgtcaggcgctatctgaagcagctcctgtttatacgatgacaccggaagacgtggaccttactggtctcgactatacgcccgttttcggatc

SEQ ID NO: 54 - DNA, 355 bp, synthetic construct
  misc_feature 1..355: note = Template
  source 1..355: mol_type = other DNA
cccatgagacatacaaaaaggtaatgccgccggcctcgtagtgaaaattaatggtgttgaacagatcgcgcaccaatacggcgtcctgcagacagtaacggccttccggaatgcaaatcagtcgtcactcactggtttcatcatatctggcgttaatggagttcagtggtaatacaatgaccagagaggcttctagggctgtcctgcgttttgtaaccgtcacagccgaagccttacggttcaggcagatacagagagaatttcgtcaggcactgtctgaaacagctcctgtttatacgatgacgccggaagacgtggacctcactggtctcgactatacgcccgttttcggatc

SEQ ID NO: 55 - DNA, 388 bp, synthetic construct
  misc_feature 1..388: note = Template
  source 1..388: mol_type = other DNA
cccatgagacatacaaaaaggtaatgccgccggcctcgtagtgaaaattaatggtgttgaacagatcgcgcaccaatacggcgtcctgcagacagtaacggcccgcagcgctggaacgctccggaatgcaaatcagccgtcactcactggtttcatcatatctggagttaatggagttccgtggtaatgccatgaccagagcggcgtccagggctgtcctgcgttttgtcactgtcacagccgaggctttacgtttcaggcaaatacagagagaatttcgtccggcgctatctgaagctgctcctgtttatacgatgacaccggaagacgtggacctcactctgaactgggggagaatcggtctcgactatacgcccgttttcggatc

SEQ ID NO: 56 - DNA, 338 bp, synthetic construct
  misc_feature 1..338: note = Template
  source 1..338: mol_type = other DNA
cccatgagacatacaaaaaggtaatgccgccggcctcgtagtgaaaattaatggtgttgaacagatcgcgcaccaatacggcgtcctgcagacagtaacggccatcagtcgtcactcactggtttcatcatatctggcgttaatggagttcagtggtaatacaatgaccagagaggcttctagggctgtcctgcgttttgtaaccgtcacagccgaagccttacggttcaggcagatacagagagaatttcgtcaggcactgtctgaaacagctcctgtttatacgatgacgccggaagacgtggacctggtctcgactatacgcccgttttcggatc

SEQ ID NO: 57 - DNA, 291 bp, synthetic construct
  misc_feature 1..291: note = Template
  source 1..291: mol_type = other DNA
cccatgagacatacaaaaaggtaatgccgccggcctcgtagtgaaaattaatggtgttgaacagatcgcgcaccaatacggcgtcctgcagacagtaacggcctcagtggtaatacaatgaccagagatgcatccagagcagttctgcgttttgtcactgtcacagcagaagccttacgcttcaggcagatacagagagaatttcgtcaggcaccgtctgaaactgctcctgtgtgcatacgatgacgccgggagacgtggaggtctcgactatacgcccgttttcggatc

SEQ ID NO: 58 - DNA, 288 bp, synthetic construct
  misc_feature 1..288: note = Template
  source 1..288: mol_type = other DNA
cccatgagacatacaaaaaggtaatgccgccggcctcgtagtgaaaattaatggtgttgaacagatcgcgcaccaatacggcgtcctgcagacagtaacggccttagtggaaatgccatgaccagagatgcatccagagcagttttgcgttttgtcactgtcacagcagaagccttacggttcaggcaaatacagagagaatttcgtctggcactgtctgaaactgctcctgtttatacgatgacaccggaagaagtggggtctcgactatacgcccgttttcggatc

SEQ ID NO: 59 - DNA, 42 bp, synthetic construct
  misc_feature 1..42: note = Microarray probe
  source 1..42: mol_type = other DNA
tctctctctctctctctctcctctctgtatttgcctgaaacg

SEQ ID NO: 60 - DNA, 45 bp, synthetic construct
  misc_feature 1..45: note = Microarray probe
  source 1..45: mol_type = other DNA
tctctctctctctctctctctacgtttcaggcaaatacagagaga

SEQ ID NO: 61 - DNA, 45 bp, synthetic construct
  misc_feature 1..45: note = Microarray probe
  source 1..45: mol_type = other DNA
tctctctctctctctctctctctctctgtatttgcctgaaacgta

SEQ ID NO: 62 - DNA, 22 bp, synthetic construct
  misc_feature 1..22: note = Microarray probe
  source 1..22: mol_type = other DNA
ctctctgtatttgcctgaaacg

SEQ ID NO: 63 - DNA, 25 bp, synthetic construct
  misc_feature 1..25: note = Microarray probe
  source 1..25: mol_type = other DNA
tctctctgtatttgcctgaaacgta